(19) United States
(12) Patent Application Publication
(10) Pub. No.: US 2007/0197658 A1
(43) Pub. Date: Aug. 23, 2007

(54) Polyamines and their use as antibacterial and sensitizing agents

(76) Inventors: Sunil A. David, Lawrence, KS (US); Apurba Dutta, Lawrence, KS (US)

Correspondence Address: STINSON MORRISON HECKER LLP;ATTN: PATENT GROUP, 1201 WALNUT STREET, SUITE 2800, KANSAS CITY, MO, 64106-2150, US

(21) Appl. No.: 11/709,425

(22) Filed: Feb. 22, 2007

Related U.S. Application Data
(60) Provisional application No. 60/775,512, filed on Feb. 22, 2006.

(51) Int. Cl. A61K 31/16; A61K 31/13

(52) U.S. Cl. 514/625; 514/674

(57) ABSTRACT

Polyamines with varying chain-lengths were evaluated for antimicrobial activity in order to test the hypothesis that these bis-cationic amphipathic compounds may also bind to and permeabilize intact Gram negative bacterial membranes. The compounds were found to possess significant antimicrobial activity and mediated via permeabilization of bacterial membranes. Homologated spermine, bis-acylated with C₈ or C₉ chains was found to profoundly sensitize E. coli to hydrophobic antibiotics such as rifampicin.

### CROSS-REFERENCE TO RELATED APPLICATIONS

This application is based on and claims priority to U.S. Provisional Application Ser. No. 60/775,512 filed on Feb. 22, 2006, which is hereby incorporated herein by reference.

### STATEMENT REGARDING FEDERALLY SPONSORED RESEARCH OR DEVELOPMENT

Not applicable.

### BACKGROUND OF THE INVENTION

The accelerated emergence of many strains of multidrug-resistant bacteria as a result of widespread use and misuse of antibiotics has mandated the urgent need for a renewed search for novel antibacterial agents and sensitizing agents. The presence of an outer membrane (“OM”) in Gram negative bacteria provides an effective protective barrier in these organisms to antimicrobial agents that may otherwise be active. For instance, it has been reported that in antibiotics of natural origin that are active against Gram positive bacteria, more than 90% lacked activity at a useful level against Gram negative E. coli. See Vaara, Antibiotic-supersusceptible mutants of Escherichia coli and Salmonella typhimurium, Antimicrob. Agents Chemother. 37:2255-2260 (1993). The barrier, formed by a divalent cation-crosslinked matrix of lipopolysaccharide (“LPS”) molecules on the outer leaflet of the OM can be breached by metal-chelating agents such as EDTA, or via displacement of LPS-bound metals by polycations of diverse structural classes. See Hancock et al., Compounds which increase the permeability of the Pseudomonas aeruginosa outer membrane, Antimicrob. Agents Chemother. 26:48-52 (1984); Osborn, Biosynthesis and assembly of the lipopolysaccharide of the outer membrane, p. 15-34; In: M. Inouye (ed.), Bacterial outer membranes. Biogenesis and functions. John Wiley & Sons, New York, Chichester, Brisbane, Toronto (1979); Rietschel et al., Bacterial endotoxin: molecular relationships of structure to activity and function, FASEB J. 8:217-225 (1994); Vaara, Agents That Increase the Permeability of the Outer Membrane, Microbiological Reviews 56:395-411 (1992); Vaara et al., Polycations as outer membrane disorganizing agents, Antimicrobial Agents and Chemotherapy 24:114-122 (1983).

Polymyxin B (“PMB”), a cyclic, penta-cationic, amphipathic peptide antibiotic, isolated from Bacillus polymyxa is a prototype membrane-perturbing agent, whose antibacterial action is manifested via its binding to the lipid A moiety of LPS. Perturbation of the OM alone has been thought to result in bacterial killing since immobilized PMB can disrupt the OM. See Rosenthal et al., Disruption of the Escherichia coli outer membrane permeability barrier by immobilized polymyxin B, The Journal of Antibiotics 30:1087-1092 (1977). However, alternate hypotheses concerning “self-promoted” uptake of the antibiotic and subsequent perturbation of the inner membrane (“IM”), culminating in bacterial lysis have also been suggested. See Devine et al., Cationic peptides: distribution and mechanisms of resistance, Curr. Pharm. Des. 8:703-714 (2002); Zhang et al., Interactions of bacterial cationic peptide antibiotics with outer and cytoplasmic membranes of Pseudomonas aeruginosa, Antimicrob. Agents Chemother. 44:3317-3321 (2000).

For a number of years, the present inventors have evaluated cationic, amphipathic small molecules as specific LPS sequestrants. See David et al., Towards a rational development of anti-endotoxin agents: novel approaches to sequestration of bacterial endotoxins with small molecules, J. Molec. Recognition 14:370-387 (2001), which is incorporated by reference. Of particular interest are lipopolyamines, generally characterized by the presence of long-chain substituents on polyamine scaffolds. Prior work has shown that some members of the lipopolyamine class bind LPS, are effective in preventing endotoxic shock in animal models, and appear to be nontoxic both in vitro and in vivo. See David et al., Lipopolyamines: novel antiendotoxin compounds that reduce mortality in experimental sepsis caused by Gram negative bacteria, Antimicrob. Agents Chemother. 43:912-919 (1999); Burns et al., U.S. Patent Application No. 2006/0122279 entitled “Hydrophobic Polyamine Amides as Potent Lipopolysaccharide Sequestrants”; Zorko, M., Combination of Antimicrobial and Endotoxin-Neutralizing Activities of Novel Oleoylamines, Antimicrob. Agents Chemother. 49:2307-2313 (2005), all of which are incorporated by reference. In particular, certain N-acylated homologated spermine compounds were recently found to sequester LPS. See Miller et al., Lipopolysaccharide Sequestrants: Structural Correlates of Activity and Toxicity in Novel Acylhomospermines, J. Med. Chem. 48:2589-2599 (2005). These lipopolyamine compounds possess potent endotoxin-sequestering activity in vitro, and afford protection in animal models of Gram negative sepsis.

Therapeutic agents with combined intrinsic antibacterial activity and endotoxin-sequestering activities may offer significant advantages in addressing the problem of antibiotic-induced endotoxin release, a contributory factor in the development of endotoxic shock in Gram negative sepsis. In the present invention, it was surprisingly demonstrated that the mono-acyl and bis-acyl homospermine compounds possess intrinsic antibacterial activity (in addition to their LPS sequestering ability previously reported). Further, these compounds surprisingly increased the permeability of the IM and OM both Gram negative and Gram positive bacteria. Thus, the present invention is directed to a new use of such compounds as sensitizing agents to be co-administered with other antibacterial agents, in particular hydrophobic antibiotics. In addition, in the present invention, novel alkyl and alkenyl analogues are synthesized.

### BRIEF SUMMARY OF THE INVENTION

In one aspect, the present invention is directed to the use of certain substituted polyamines as therapeutics which possess intrinsic antibacterial activity. Pharmaceutical composition comprising such compounds are also provided.

In another aspect, the present invention is directed to the use of certain substituted polyamines as sensitizing agents for increasing the susceptibility of a bacterium to an antibacterial agent. In a preferred aspect, the polyamines are naturally occurring or synthetic polyamine containing between 4 and 8 amino groups (preferably 5 to 6 amino groups). Such polyamines may be derived, for example, form cadaverine, putrescine, spermidine, spermine, and the like. The polyamines are preferably substituted with at least one functional group selected from a C₇ to C₃₀ alkyl, C₇ to C₃₀ alkenyl, or C₇ to C₃₀ acyl. In exemplary aspect, homologated spermine, bis-acylated with C₈ or C₉ chains was found to profoundly sensitize E. coli to hydrophobic antibiotics such as rifampicin.

In a preferred aspect, the substituted polyamines of the present invention also possess the ability to sequester LPS in vitro, and still more preferably in vivo using an applicable animal model.

In am even more preferred aspect, the substituted polyamines of the present invention are useful as antibacterial agents having intrinsic antibacterial activity, sensitizers of bacteria to other antibiotics, and disrupters of bacterial membranes.

Pharmaceutical compositions comprising the substituted polyamines of the present invention can be used to treat humans and animals having a bacterial infection. The pharmaceutical compositions can include an effective amount of the polyamine compounds of the present invention alone or in combination with other antibacterial agents.

Yet another aspect of the present invention is to provide methods for treating mammals suffering from infections caused by Gram negative bacteria, and/or from one or more clinical consequences of such infections (e.g., septic shock).

A further aspect of the present invention is to provide a method for increasing the permeability of the OM of Gram negative bacteria.

A further aspect of the present invention is to provide a method for increasing the permeability of the IM of Gram negative bacteria and the membrane of Gram positive bacteria.

A still further aspect of the present invention is to increase the effectiveness of Gram negative bactericidal agents.

In yet another aspect, the substituted polyamines exhibit significant antibacterial activity in the presence of physiological concentrations of human serum albumin.

Without wishing to be bound to any particular theory, the polyamine compounds of the present invention also act to sensitize bacteria to other antibiotics. The compounds cause bacteria to become more susceptible to other antibiotics by increasing the permeability of the OM of the bacteria. Measurements used to quantitate the effects of the compounds on bacteria include measurement of minimum inhibitory concentrations (“MICs”), measurement of minimum bactericidal concentrations (“MBCs”) and the ability of the substituted polyamines to lower the MICs of other antibiotics, e.g., rifampin, erythromycin, and/or novobiocin.

Additional aspects of the invention, together with the advantages and novel features appurtenant thereto, will be set forth in part in the description which follows, and in part will become apparent to those skilled in the art upon examination of the following, or may be learned from the practice of the invention. The objects and advantages of the invention may be realized and attained by means of the instrumentalities and combinations particularly pointed out in the appended claims.

### BRIEF DESCRIPTION OF THE DRAWINGS

FIG. 1A shows the correlation of MICs of the acylpolyamines against E. coli (ATCC 9637) and S. aureus (ATCC 13709). MICs were determined by broth microdilution method in Mueller-Hinton broth as per NCCLS guidelines. FIG. 1B shows the correlation of OM and IM permeabilizing activity. OM permeabilizing activity was determined using E. coli ML-35p (parent i⁻ z⁺ y⁻ ATCC 43827 transformed with pBR322 vector encoding periplasmic β-lactamase); the leakage of periplasmic β-lactamase activity was quantified using nitrocefin as a chromogenic substrate. IM permeabilizing activity was determined using E. coli ML-35 using o-nitrophenyl-β-D-galactopyranoside as the substrate to determine the β-galactosidase activity. In all experiments, PMB, PMBN, and melittin were used as reference compounds.

FIG. 2A is shows the data from a representative titration experiment showing the sensitization activities of some bis-acyl analogues. E. coli ATCC 9637 was seeded in MH broth in chequerboard format in a 384-well plate containing a constant concentration of compound and varying doses of rifampicin. Bacterial growth was monitored by turbidimetry at 600 nm. PMBN and melittin were used as positive controls. Wells containing no test-compound served as negative control. FIGS. 2B and 2C are plot of OM and IM permeabilization activity against extent of sensitization by the acylpolyamines. Fold sensitization was calculated as MIC_{Rifampicin alone}/MIC_{Rifampicin+10μM Compound}.

FIG. 3 shows the correlation of MIC against S. aureus (FIG. 3A) and E. coli (FIG. 3B) with length of the acyl group.

FIG. 4A shows the surface tension measurements of 4 and 8 series compounds by dynamic pressure tensiometry. The slopes of the lines are directly proportional to the critical micellar concentrations. FIGS. 4B and 4C shows the correlation of surface activity with antimicrobial activities against E. coli ATCC 9637, and S. aureus ATCC 13709.

FIG. 5A shows the hemolytic activity of the acylpolyamines in a highly dilute, washed, aged, human erythrocytes suspended in isotonic saline quantified by automated video microscopy. FIG. 5B shows the correlation of carbon number of 4 and 8 series of compounds with hemolytic activity. FIG. 5C shows abrogation of hemolysis as described above by representative mono-acyl compounds in the presence of 650 μM of human serum albumin. FIG. 5D shows the absorptimetric determination of hemolytic activity in fresh, whole human blood by quantifying released hemoglobin. Melittin was used as a positive control.

FIG. 6 shows the MICs of 8b with and without physiological concentration of HSA. A stock solution of 8b was serially diluted in either a 4.5 g/100 ml solution of sterile-filtered HSA, or sterile, distilled water. An equal volume of a suspension of either E. coli ATCC 9637 (FIG. 6A) or S. aureus ATCC 13709 (FIG. 6B) in 2× Mueller-Hinton broth was added using an automated liquid dispensing system to a 384 well plate, and bacterial growth was measured by absorptimetry as described in Materials and Methods. Also shown is the MIC of amoxicillin with or without HSA, as an internal control.

FIG. 7A shows the Interaction of the mono-acylated polyamine 4e with human serum albumin as probed by isothermal titration calorimetry; a single-site model yielded a stoichiometry of 5:1 of 4e:HSA with a k_{D} of ˜2 μM. FIG. 7B shows the inhibition of hemolysis of mono-acylated polyamine 4e by HSA. FIG. 7C shows the identical potency of NO inhibition of mono-acylated polyamine 4e solubilized in DMSO or in HSA.

### DETAILED DESCRIPTION OF PREFERRED EMBODIMENT

The present invention is directed to the use of certain substituted polyamines and their pharmaceutically acceptable salts as therapeutics which possess intrinsic antibacterial activity. The substituted polyamines may also be used as sensitizing agents for increasing the susceptibility of a bacterium to an antibacterial agent. The substituted polyamines are characterized according to Formula 1:

R¹-polyamine-R²

wherein “polyamine” refers to any naturally occurring or synthetic polyamine, preferably those containing between 4 and 8 amino groups;

wherein R¹ and R² are independently hydrogen, C₇ to C₃₀ alkyl, C₇ to C₃₀ alkenyl, or C₇ to C₃₀ acyl; and wherein at least one of R¹ and R² is not hydrogen.

In a further aspect, the compounds used in the methods of the present invention may be characterized according to Formula 2:

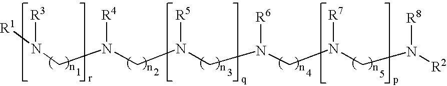

wherein R¹ and R² are independently hydrogen, C₇ to C₃₀ alkyl, C₇ to C₃₀ alkenyl, or C₇ to C₃₀ acyl; and wherein at least one of R¹ and R² is not hydrogen;

wherein R³, R⁴, R⁵, R⁶, R⁷, and R⁸ are independently hydrogen or lower alkyl;

wherein n₁, n₂, n₃, n₄, and n₅ are independently 1, 2, 3, 4, 5, 6, 7, 8, 9, or 10; and

and wherein p, q, and r are independently 0, 1, 2, 3, 4, or 5.

In another aspect, the compounds used in the methods of the present invention are polyamines having 5 amino groups according to Formula 3:

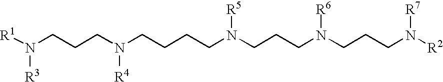

wherein R¹ and R² are independently hydrogen, C₇ to C₃₀ alkyl, C₇ to C₃₀ alkenyl, or C₇ to C₃₀ acyl; and wherein at least one of R¹ and R² is not hydrogen; and

wherein R³, R⁴, R⁵, R⁶, and R⁷ are independently hydrogen or lower alkyl.

Acyl Polyamines (Penta-Amino Polyamines)

In another aspect, the compounds used in the methods of the present invention are polyamines having 5 amino groups according to Formula 4A:

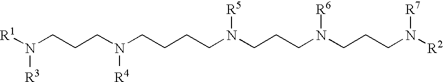

wherein R¹ and R² are independently acyl and selected from the group consisting of —COC₈H₁₇, —COC₉H₁₉, —COC₁₀H₂₁, —COC₁₁H₂₃, —COC₁₂H₂₅, —COC₁₃H₂₇, —COC₁₄H₂₉, —COC₁₅H₃₁, —COC₁₆H₃₃, —COC₁₇H₃₅, and —COC₁₈H₃₇; and

wherein R³, R⁴, R⁵, R⁶, and R⁷ are independently hydrogen or lower alkyl, and preferably hydrogen or methyl.

In still another aspect, the compounds used in the methods of the present invention are polyamines having 5 amino groups according to Formula 4B:

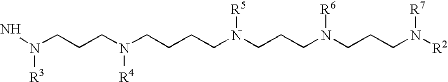

and wherein R² is an acyl selected from the group consisting of —COC₈H₁₇, —COC₉H₁₉, —COC₁₀H₂₁, —COC₁₁H₂₃, —COC₁₂H₂₅, —COC₁₃H₂₇, —COC₁₄H₂₉, —COC₁₅H₃₁, —COC₁₆H₃₃, —COC₁₇H₃₅, and —COC₁₈H₃₇; and

wherein R³, R⁴, R⁵, R⁶, and R⁷ are independently hydrogen or lower alkyl.

In still another aspect, the compounds used in the methods of the present invention are defined according to Formula 4C:

NH₂C₃H₆NHC₄H₈NHC₃H₆NHC₃H₆NH—CO(CH₂)ₓCH₃

wherein x is an integer between 7 and 25, more preferably between 10 and 18, and still more preferably between 12-16.

In still another aspect, the compounds used in the methods of the present invention are defined according to Formula 4D:

CH₃(CH₂)ₓCO—NHC₃H₆NHC₄H₈NHC₃H₆NHC₃H₆NH—CO(CH₂)ₓCH₃

wherein x is an integer between 7 and 25, more preferably between 8-12.

Alkyl Polyamines (Penta-amino Polyamines)

In another aspect, the compounds used in the methods of the present invention are polyamines having 5 amino groups are characterized according to Formula 5A:

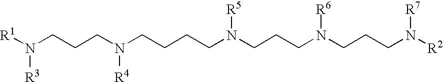

and wherein R² is a C₇ to C₃₀ alkyl, more preferably a C₁₂ to C₂₀ alkyl, and most preferably a C₁₅ to C₁₇ alkyl; and

wherein R³, R⁴, R⁵, R⁶, and R⁷ are independently hydrogen or lower alkyl, and preferably hydrogen or methyl.

In still another aspect, the compounds used in the methods of the present invention are polyamines having 5 amino groups according to Formula 5B:

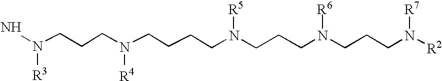

and wherein R² is a C₇ to C₃₀ alkyl, more preferably a C₁₂ to C₂₀ alkyl, and most preferably a C₁₅ to C₁₇ alkyl; and

wherein R³, R⁴, R⁵, R⁶, and R⁷ are independently hydrogen or lower alkyl, preferably hydrogen or methyl.

In still another aspect, the compounds used in the methods of the present invention are defined according to Formula 5C:

NH₂C₃H₆NHC₄H₈NHC₃H₆NHC₃H₆NH—R²

wherein R² is a C₇ to C₃₀ alkyl, more preferably a C₁₂ to C₂₀ alkyl, and most preferably a C₁₅ to C₁₇ alkyl.

In still another aspect, the compounds used in the methods of the present invention are defined according to Formula 5D:

CH₃(CH₂)ₓ—NHC₃H₆NHC₄H₈NHC₃H₆NHC₃H₆NH—CH₂)ₓCH₃

wherein x is an integer between 7 and 29, more preferably between 8-24, and still more preferably between 10 and 18.

In still another aspect, the compounds used in the methods of the present invention are defined according to Formula 5E:

NH₂C₃H₆NHC₄H₈NHC₃H₆NHC₃H₆NH—(CH₂)ₓCH₃

wherein x is an integer between 7 and 29, more preferably between 8-24, and still more preferably between 10 and 18 wherein x is an integer between 7 and 25, more preferably between 8-12. In an exemplary aspect, the compound is NH₂C₃H₆NHC₄H₈NHC₃H₆NHC₃H₆NHC₁₆H₃₃ (DS-96).

Alkenyl Polyamines(Penta-Amino Polyamines)

In another aspect, the compounds used in the methods of the present invention are nes having 5 amino groups according to Formula 6A:

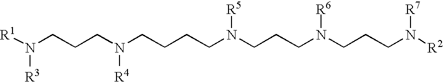

and wherein R² is a C₁₇ to C₃₀ alkenyl, more preferably a C₁₂ to C₂₀ alkenyl, and most preferably a C₁₅ to C₁₇ alkenyl; and

wherein R³, R⁴, R⁵, R⁶, and R⁷ are independently hydrogen or lower alkyl.

In still another aspect, the compounds used in the methods of the present invention are polyamines having 5 amino groups according to Formula 6B:

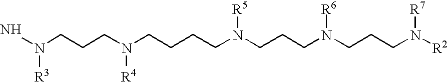

and wherein R² is a C₇ to C₃₀ alkenyl, more preferably a C₁₂ to C₂₀ alkenyl, and most preferably a C₁₅ to C₁₇ alkenyl; and

wherein R³, R⁴, R⁵, R⁶, and R⁷ are independently hydrogen or lower alkyl, preferably hydrogen or methyl.

In still another aspect, the compounds used in the methods of the present invention are defined according to Formula 6C:

NH₂C₃H₆NHC₄H₈NHC₃H₆NHC₃H₆NH—R²

wherein R² is a C₇ to C₃₀ alkenyl.

The alkenyl is preferably branched such that there are at least two relatively long hydrophobic chains. Thus, in another aspect, the compounds used in the methods of the present invention are defined by Formula 6D:

NH₂C₃H₆NHC₄H₈NHC₃H₆NHC₃H₆NH—(CH₂)C(R¹⁰)═CHR¹¹

wherein R¹⁰ and R¹¹ are independently C₇ to C₂₀ alkyl, preferably C₁₂ to C₁₈ alkyl, and more preferably C₁₄ to C₁₆ alkyl. An exemplary polyamine having an alkenyl side chain is NH₂C₃H₆NHC₄H₈NHC₃H₆NHC₃H₆NHCH₂C(C₁₄H₂₉)═C₁₆H₃₂ (EVK-203).

In still another aspect, the compounds used in the methods of the present invention are defined according to Formula 6E:

R¹¹═CHC(R¹⁰)CH₂—NH₂C₃H₆NHC₄H₈NHC₃H₆NHC₃H₆NH—(CH₂)C(R¹⁰)═CHR¹¹

wherein R¹⁰ and R¹¹ are independently C₇ to C₂₀ alkyl, preferably C₁₂ to C₁₈ alkyl, and more preferably C₁₄ to C₁₆ alkyl.

Acyl Polyamines (Hexa-Amino Polyamines)

In another aspect, the compounds used in the methods of the present invention are polyamines having 6 amino groups according to Formula 7A:

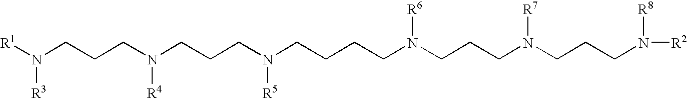

wherein R¹ and R² are independently hydrogen, C₇ to C₃₀ alkyl, C₇ to C₃₀ alkenyl, or C₇ to C₃₀ acyl; and wherein at least one of R¹ and R² is not hydrogen; and

wherein R³, R⁴, R⁵, R⁶, R⁷, and R⁸ are independently hydrogen or lower alkyl.

In another aspect, the compounds used in the methods of the present invention are polyamines having 6 amino groups according to Formula 7B:

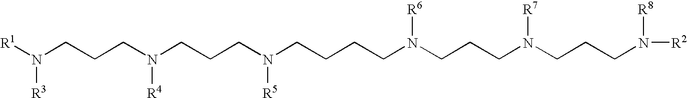

wherein R¹ and R² are independently acyl and selected from the group consisting of —COC₈H₁₇, —COC₉H₁₉, —COC₁₀H₂₁, —COC₁₁H₂₃, —COC₁₂H₂₅, —COC₁₃H₂₇, —COC₁₄H₂₉, —COC₁₅H₃₁, —COC₁₆H₃₃, —COC₁₇H₃₅, and —COC₁₈H₃₇; and

wherein R³, R⁴, R⁵, R⁶, R⁷ and R⁸ are independently hydrogen or lower alkyl.

In another aspect, the compounds used in the methods of the present invention are polyamines having 6 amino groups according to Formula 7C:

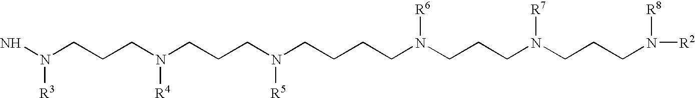

and wherein R² is an acyl selected from the group consisting of —COC₈H₁₇, —COC₉H₁₉, —COC₁₀H₂₁, —COC₁₁H₂₃, —COC₁₂H₂₅, —COC₁₃H₂₇, —COC₁₄H₂₉, —COC₁₅H₃₁, —COC₁₆H₃₃, —COC₁₇H₃₅, and —COC₁₈H₃₇.

In still another aspect, the compounds used in the methods of the present invention are defined according to Formula 7D:

NHC₃H₆NHC₃H₆NHC₄H₈NHC₃H₆NHC₃H₆NH—CO(CH₂)ₓCH₃

wherein x is an integer between 7 and 29, more preferably between 10 and 17, and still more preferably between 12 and 16.

In still another aspect, the compounds used in the methods of the present invention are defined according to Formula 7E:

CH₃(CH₂)ₓCO—NHC₃H₆NHC₃H₆NHC₄H₈NHC₃H₆NHC₃H₆NH—CO(CH₂)ₓCH₃

wherein x is an integer between 7 and 25, more preferably between 8-12.

Alkyl Polyamines (Hexa-Amino Polyamines)

In another aspect, the compounds used in the methods of the present invention are polyamines having according to Formula 8A:

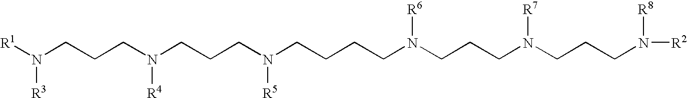

and wherein R² is a C₇ to C₃₀ alkyl, more preferably a C₁₂ to C₂₀ alkyl, and most preferably a C₁₅ to C₁₇ alkyl; and

wherein R³, R⁴, R⁵, R⁶, R⁷ and R⁸ are independently hydrogen or lower alkyl, preferably hydrogen or methyl.

In another aspect, the compounds used in the methods of the present invention are polyamines having 6 amino groups; n₃ is 2; n₁, n₂, n₄, and n₅ are 1; and p is 1, and wherein R¹ is hydrogen such that the compounds are characterized according to Formula 8B:

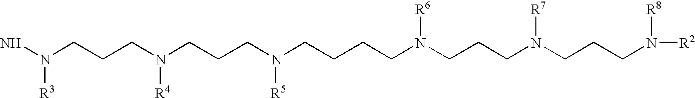

and wherein R² is a C₇ to C₃₀ alkyl, more preferably a C₁₂ to C₂₀ alkyl, and most preferably a C₁₅ to C₁₇ alkyl; and

wherein R³, R⁴, R⁵, R⁶, R⁷ and R⁸ are independently hydrogen or lower alkyl, preferably hydrogen or methyl.

In still another aspect, the compounds used in the methods of the present invention are defined according to Formula 8C:

NH₂C₃H₆NHC₃H₆NHC₄H₈NHC₃H₆NHC₃H₆NH—R²

wherein R² is a C₇ to C₃₀ alkyl, more preferably a C₁₂ to C₂₀ alkyl, and most preferably a C₁₅ to C₁₇ alkyl.

In still another aspect, the compounds are defined according to Formula 8D:

NH₂C₃H₆NHC₃H₆NHC₄H₈NHC₃H₆NHC₃H₆NH—CH₂)ₓCH₃

wherein x is an integer between 7 and 29, more preferably between 10 and 17, more preferably between 12 and 16.

In still another aspect, the compounds used in the methods of the present invention are defined according to Formula 8E:

CH₃(CH₂)ₓ—NHC₃H₆NHC₃H₆NHC₄H₈NHC₃H₆NHC₃H₆NH—(CH₂)ₓCH₃

wherein x is an integer between 7 and 29, more preferably between 10 and 17, and still more preferably between 12 and 16.

Alkenyl Polyamines (Hexa-Amino Polyamines)

In another aspect, the compounds used in the methods of the present invention are polyamines having 6 amino groups according to Formula 9A:

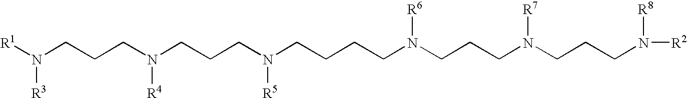

and wherein R² is a C₇ to C₃₀ alkenyl, more preferably a C₁₂ to C₂₀ alkenyl, and most preferably a C₁₅ to C₁₇ alkenyl; and

wherein R³, R⁴, R⁵, R⁶, R⁷ and R⁸ are independently hydrogen or lower alkyl, preferably hydrogen or methyl.

In another aspect, the compounds used in the methods of the present invention are polyamines having 6 amino groups according to Formula 9B:

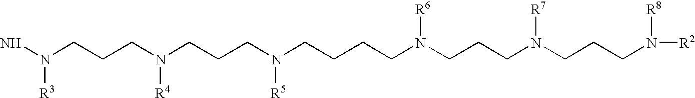

and wherein R² is a C₇ to C₃₀ alkenyl, more preferably a C₁₂ to C₂₀ alkenyl, and most preferably a C₁₅ to C₁₇ alkenyl; and

wherein R³, R⁴, R⁵, R⁶, R⁷ and R⁸ are independently hydrogen or lower alkenyl, preferably hydrogen or methyl.

In still another aspect, the compounds used in the methods of the present invention are defined according to Formula 9C:

NH₂C₃H₆NHC₃H₆NHC₄H₈NHC₃H₆NHC₃H₆NH—R²

wherein R² is a C₇ to C₃₀ alkenyl, more preferably a C₁₂ to C₂₀ alkenyl, and most preferably a C₁₅ to C₁₇ alkenyl.

In still another aspect, the compounds used in the methods of the present invention are defined according to Formula 9D:

NH₂C₃H₆NHC₃H₆NHC₄H₈NHC₃H₆NHC₃H₆NH—(CH₂)C(R¹⁰)═CHR¹¹

wherein R¹⁰ and R¹¹ are independently C₇ to C₂₀ alkyl, preferably C₁₂ to C₁₈ alkyl, and more preferably C₁₄ to C₁₆ alkyl.

In still another aspect, the compounds used in the methods of the present invention are defined according to Formula 9E:

R¹¹═CHC(R¹⁰)CH₂—NHC₃H₆NHC₃H₆NHC₄H₈NHC₃H₆NHC₃H₆NH—(CH₂)C(R¹⁰)═CHR¹¹

wherein R¹⁰ and R¹¹ are independently C₇ to C₂₀ alkyl, preferably C₁₂ to C₁₈ alkyl, and more preferably C₁₄ to C₁₆ alkyl.

In addition, the present invention is directed to pharmaceutical compositions comprising a therapeutically effective amount of one of the foregoing polyamines together with an antibacterial agent.

Further, apart from anti-microbial action, the permeability provided by the compounds may enhance introduction of a great variety of substances into microbes. For example, the compounds may be used to enhance introduction of macromolecules such as DNA or RNA into microbes, particularly Gram negative bacteria. In that case, there may be no need for the traditional vectors (e.g., phages) used to package nucleic acids when transfecting the microbes. Conditions and techniques for introducing such macromolecules into microbes using the compounds of the invention will in most cases be routine.

In still a further aspect, novel polyamine compounds which are alkyl and alkenyl derivatives are provided. In one aspect, the compounds are defined according to:

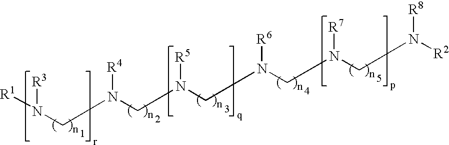

wherein R¹ and R² are independently hydrogen, C₇ to C₃₀ alkyl or C₇ to C₃₀ alkenyl; and wherein at least one of R¹ and R² is not hydrogen;

wherein R³, R⁴, R⁵, R⁶, R⁷, and R⁸ are independently hydrogen or lower alkyl;

wherein n₁, n₂, n₃, n₄, and n₅ are independently 1, 2, 3, 4, 5, 6, 7, 8, 9, or 10; and

and wherein p, q, and r are independently 0, 1, 2, 3, 4, or 5.

Definitions

Molecular terms, when used in this application, have their common meaning unless otherwise specified. It should be noted that the alphabetical letters used in the formulas of the present invention should be interpreted as the functional groups, moieties, or substituents as defined herein. Unless otherwise defined, the symbols will have their ordinary and customary meaning to those skilled in the art.

As used herein, the term “between” in the context of an integer is inclusive of the limits of the range. For example, the term “between 10 and 15” includes the integers 10 and 15.

As used herein, the term “C₇ to C₃₀ alkyl” refers to a straight or branched saturated hydrocarbon group of 7 to 30 carbon atoms. Examples for alkyl groups containing up to 30 carbon atoms include eicosyl, heneicosyl, docosyl, tricosyl, tetracosyl, pentacosyl, hexacosyl, heptacosyl, octacosyl, nonacosyl, and triacontyl.

As used herein, the term “lower alkyl” denotes an alkyl group of 1-7 carbons, preferably 1-4 carbons, for example methyl, ethyl, propyl, isopropyl, butyl, and isomers thereof. Most preferably, the lower alkyl is a methyl group.

As used herein, the term “C₇ to C₃₀ alkenyl” refers to unsaturated groups analogous in length and possible substitution to the alkyls described above, but that contain at least one double bond. The group may be polyunsaturated and have multiple double bonds, e.g. 2, 3, 4, 5, 6, etc. double bonds. The unsaturation (i.e. —CH══CH—) may occur at any position along the carbon chain.

As used herein, the term “C₇ to C₃₀ acyl” refers to the group —COR′, wherein R′ is a C₇ to C₃₀ alkyl or C₇ to C₃₀ alkenyl.

As used herein, the term “administration” refers to a method of giving a dosage of pharmaceutical composition comprising one of the polyamines of the present invention to a mammal, where the method is, e.g., topical, oral, intravenous, transdermal, intraperitoneal, or intramuscular. The preferred method of administration can vary depending on various factors, e.g., the components of the pharmaceutical composition, the site of the potential or actual bacterial infection, the bacterium involved, and the severity of an actual bacterial infection.

As used herein, the term “antibacterial agent” refers to both naturally occurring antibiotics produced by microorganisms to suppress or inhibit the growth of other microorganisms, and agents synthesized or modified in the laboratory which have either bactericidal or bacteriostatic activity, e.g., beta-lactam antibacterial agents including, e.g., ampicillin, cloxacillin, oxacillin, and piperacillin, cephalosporins and other cephems including, e.g., cefaclor, cefamandole, cefazolin, cefoperazone, cefotaxime, cefoxitin, ceftazidime, ceftriaxone, and cephalothin; carbapenems including, e.g., imipenem and meropenem; and glycopeptides, macrolides, quinolones, tetracyclines, and aminoglycosides. In general, if an antibacterial agent is “bacteriostatic,” it means that the agent essentially stops bacterial cell growth (but does not kill the bacteria); if the agent is “bactericidal,” it means that the agent kills the bacterial cells (and may stop growth before killing the bacteria). In general, antibiotics and similar agents accomplish their anti-bacterial effect through several mechanisms of action which can be generally grouped as follows: (I) agents acting on the bacterial cell wall such as bacitracin, the cephalosporins, cycloserine, fosfomycin, the penicillins, ristocetin, and vancomycin; (2) agents affecting the cell membrane or exerting a detergent effect, such as colistin, novobiocin and polymyxins; (3) agents affecting cellular mechanisms of replication, information transfer, and protein synthesis by their effects on ribosomes, e.g., the aminoglycosides, the tetracyclines, chloramphenicol, clindamycin, cycloheximide, fucidin, lincomycin, puromycin, rifampicin, other streptomycins, and the macrolide antibiotics such as erythromycin and oleandomycin; (4) agents affecting nucleic acid metabolism, e.g., the fluoroquinolones, actinomycin, ethambutol, 5-fluorocytosine, griseofulvin, rifamycins; and (5) drugs affecting intermediary metabolism, such as the sulfonamides, trimethoprim, and the tuberculostatic agents isoniazid and para-aminosalicylic acid. Some agents may have more than one primary mechanism of action, especially at high concentrations. In addition, secondary changes in the structure or metabolism of the bacterial cell often occur after the primary effect of the antimicrobial drug. Preferred antibiotics include beta-lactams (penicillins and cephalosporins), vancomycins, bacitracins, macrolides (erythromycins), lincosamides (clindomycin), chloramphenicols, tetracyclines, aminoglycosides (gentamicins), amphotericins, cefazolins, clindamycins, mupirocins, sulfonamides and trimethoprim, rifampicins, metronidazoles, quinolones, novobiocins, polymixins, Gramicidins or any salts or variants thereof. Tetracyclines include, but are not limited to, immunocycline, chlortetracycline, oxytetracycline, demeclocycline, methacycline, deoxycycline, and minocycline.

As used herein, the term “bacteria” refers to all bacterial organisms, including but not limited to both Gram positive and Gram negative bacteria. Examples of Gram negative bacteria include the following species: Acidaminococcus, Acinetobacter, Aeromonas, Alcaligenes, Bacteroides, Bordetella, Branhamella, Brucella, Calymmatobacterium, Campylobacter, Cardiobacterium, Chromobactenum, Citrobacter, Edwardsiella, Enterobacter, Eschenchia, Flavobacterium, Francisella, Fusobacterium, Haemophilus, Klebsiella, Legionella, Moraxella, Morganella, Neisseria, Pasturella, Plesiomonas, Proteus, Providencia, Pseudomonas, Salmonella, Serratia, Shigella, Streptobacillus, Veillonella, Vibrio, and Yersinia species. Examples of Gram-positive bacteria include the Staphylococcus, Streptococcus, Actinomyces, and Clostridium species.

Further, while it will be appreciated that while primarily targeted at classical Gram negative staining bacteria whose outer capsule contains a substantial amount of lipid A, the polyamine compounds of the present invention may also be effective against other organisms with a hydrophobic outer capsule. For example, Mycobacterium spp. have a waxy protective outer coating, and compounds of the invention in combination with antibiotics may provide enhanced effectiveness against Mycobacterial infection, including tuberculosis. In that case, the compounds could be administered nasally (aspiration), by any of several known techniques.

As used herein, the term “bacterial infection” refers to the invasion of the host mammal by pathogenic bacteria, specifically including an invasion by bacteria resistant to one or more antibacterial agents (e.g., bacteria resistant to penicillins). This includes the excessive growth of bacteria which are normally present in or on the body of a mammal. More generally, a bacterial infection can be any situation in which the presence of a bacterial population(s) is damaging to a host mammal. Thus, a mammal is “suffering” from a bacterial infection when excessive numbers of a bacterial population are present in or on a mammal's body, or when the effects of the presence of a bacterial population(s) is damaging the cells or other tissue of a mammal.

As used herein, “concurrent administration,” “co-administration” or “co-treatment” includes administration of the agents together, or before or after each other. The polyamine compounds of the present invention and antibacterial agents (e.g. antibiotics) may be administered by different routes. For example, the acylpolyamines, alkylpolyamines, and akenylpolyamines may be administered intravenously while the antibiotics are administered intramuscularly, intravenously, subcutaneously, orally or intraperitoneally. Further, the acylpolyamines, alkylpolyamines, and akenylpolyamines and antibiotics may be given sequentially in the same intravenous line, after an intermediate flush, or may be given in different intravenous lines. The acylpolyamines, alkylpolyamines, and akenylpolyamines may be administered simultaneously or sequentially, as long as they are given in a manner sufficient to allow both agents to achieve effective concentrations at the site of infection.

As used herein, the term “inhibit” or “inhibiting” refers to a statistically significant and measurable reduction in activity, preferably as measured by one or more of the assays discussed herein, preferably a reduction of at least about 50% or more, still more preferably a reduction of about 60%, 70%, 80%, 90%, 95%, 97%, or more.

As used herein, “intrinsic antibacterial activity” refers to the effect of a compound on inhibiting the growth of a bacterium in an appropriate medium with no other antibacterial agent present. As described above, this activity can be determined by comparing the growth of the bacterium in the presence and absence of the test compound in a growth medium which is otherwise the same. The intrinsic activity may be either bacteriostatic or bactericidal activity.

As used herein, the term “sensitizing agent” refers to a compound which enhances the antibacterial activity of an antibacterial agent when co-administered that other antibacterial agent. The sensitizing agent may have intrinsic antibacterial activity and have a synergistic effect, preferably more than additive, when co-administered with the antibacterial agent. In addition, the sensitizing agent may operate as a potentiator such that while the sensitizing agent exhibits little or no antibacterial activity when used alone, the sensitizing agent can induce susceptibility to an antibacterial agent in a bacterium, especially one that is resistant to that antibacterial agent when the potentiator is used in conjunction with the antibacterial agent.

The term “MIC” refers to the lowest drug concentration that completely inhibits bacterial growth in vitro.

The “patient” or “subject” to be treated with the polyamine compounds of the present invention can be any animal, and is preferably a mammal, such as a domesticated animal or a livestock animal. More preferably, the patient is a human.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject compounds from one organ, or portion of the body, to another organ, or portion of the body. Each carrier must be “acceptable” in the sense of being compatible with the other ingredients of the formulation and not injurious to the patient. Some examples of materials which may serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. In a preferred aspect, the compounds of the present invention are complexed with a serum protein, such as albumin (human, bovine, equine, etc.).

As used herein, the term “pharmaceutically acceptable salts” refers to the relatively non-toxic, inorganic, and organic acid addition salts of compounds of the present invention. These can be prepared in situ during the final isolation and purification of the compounds of the invention, or by separately reacting a purified compound of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts, and the like. (See, for example, Berge, et al., J. Pharm. Sci. 66: 1-19 (1977)).

As use herein, a “potentiator” generally refers to a compound which enhances the antibacterial effect of an antibacterial agent when the two compounds are used in combination, but does not have significant antibacterial activity when used alone at concentrations similar to its concentration in the combination use.

As used herein, the term “therapeutically effective amount” or “pharmaceutically effective amount” is meant amounts of a compound of the present invention and optionally an antibacterial agent, as disclosed for this invention, which have a “therapeutic effect,” which generally refers to the inhibition, to some extent, of the normal metabolism of bacterial cells causing or contributing to a bacterial infection. The doses of the polyamine compounds of the present invention and optional antibacterial agent which are useful in combination as a treatment are “therapeutically effective” amounts. Thus, as used herein, a “therapeutically effective amount” means those amounts of the polyamine compounds of the present invention and antibacterial agent, which, when used in combination produce the desired therapeutic effect as judged by clinical trial results and/or model animal infection studies. In particular, embodiments, the polyamine compounds of the present invention and antibacterial agent are combined in predetermined proportions, and thus the “therapeutically effective amount” would be an amount of the combination. This amount, and the amounts of the sensitizing agent and antibacterial agent individually, can be routinely determined by one skilled in the art and will vary depending upon several factors such as the particular bacterial strain involved, and the particular sensitizing agent and antibacterial agent used. This amount can further depend on the patient's height, weight, sex, age, and medical history.

As used herein, the term “treating” refers to administering a pharmaceutical composition for prophylactic and/or therapeutic purposes. The term “prophylactic treatment” refers to treating a patient who is not yet infected, but who is susceptible to, or otherwise at risk, of a particular infection. The term “therapeutic treatment” refers to administering treatment to a patient already suffering from an infection. Thus, in preferred embodiments, treating is the administration to a mammal (either for therapeutic or prophylactic purposes) of therapeutically effective amounts of the polyamine compounds of the present invention and optionally an antibacterial agent in combination (e.g., either simultaneously or serially).

Compositions

The present invention is also directed to a composition comprising a therapeutically effective amount of the polyamines of the present invention having intrinsic antibacterial activity and one or more pharmaceutically or therapeutically acceptable carriers. In another aspect, the present invention is also directed to a composition comprising a therapeutically effective amount of the polyamines of the present invention having LPS sequestration activity and one or more pharmaceutically or therapeutically acceptable carriers. In another aspect, the present invention is also directed to a composition comprising a therapeutically effective amount of the polyamines of the present invention as a sensitizing agent and one or more pharmaceutically or therapeutically acceptable carriers. A preferred carrier in these pharmaceutically acceptable carriers is albumin. Typically, a physiological concentration of about 5-7 g per 100 ml albumin in a sterile isotonic solution is used. In addition, the polyamines of the present invention may be pre-complexed with albumin and the reconstituted for intravascular administration.

In addition, the polyamines of the present invention may be combined with other antibacterial agents. Thus, in another aspect, the compositions of the present invention preferably contain at least one sensitizing agent together with an antibacterial agent and one or more pharmaceutically acceptable carriers. The sensitizing agent antibacterial agent are in such amounts and relative proportion that the combination constitutes a pharmaceutically or therapeutically effective dose or amount. The compounds can be prepared as pharmaceutically acceptable salts (i.e., non-toxic salts which do not prevent the compound from exerting its toxicity).

The compositions may be formulated for any route of intravascular or extravascular route of administration, in particular for oral, rectal, transdermal, subcutaneous, intravenous, intramuscular, or intranasal administration. The compositions may be formulated in any conventional form, for example, as tablets, capsules, caplets, solutions, suspensions, dispersions, syrups, sprays, gels, suppositories, patches, and emulsions.

In Vitro Applications

Because the polyamine compounds of the present invention exhibit intrinsic antibacterial activity, they may be used in vitro. In addition, as sensitizing agents, the polyamine compounds of the present invention may be used in vitro together with antibacterial agents in tissue culture media to prevent contamination of eukaryotic cell cultures with bacterial, especially antibacterial-agent resistant bacteria such as MRSA.

Pharmaceutical Applications

The compositions containing the sensitizing agents can be administered for prophylactic and/or therapeutic treatments. In therapeutic applications, the compositions are administered to a patient already suffering from an infection from bacteria in an amount sufficient to cure or at least partially arrest the symptoms of the infection. In prophylactic applications, compositions containing the compounds of the invention are administered to a patient susceptible to, or otherwise at risk of, a particular infection.

Once improvement of the patient's conditions has occurred, a maintenance dose is administered if necessary. Subsequently, the dosage or the frequency of administration, or both, can be reduced, as a function of the symptoms, to a level at which the improved condition is retained. When the symptoms have been alleviated to the desired level, treatment can cease. Patients can, however, require intermittent treatment on a long-term basis upon any recurrence of the disease symptoms.

Examples are provided below to illustrate various aspects and embodiments of the present invention. These examples are not intended in any way to limit the disclosed invention.

### EXAMPLE 1

### Synthesis of Acylpolyamines

The details of the syntheses of the acylpolyamines (except 4g) have recently been published Miller et al., Lipopolysaccharide Sequestrants: Structural Correlates of Activity and Toxicity in Novel Acylhomospermines, J. Med. Chem. 48:2589-2599 (2005), which is incorporated by reference.

A summary of the synthetic strategy and the structures of the mono- and bis-acyl compounds are shown in the scheme below. Compound 4g was characterized by NMR spectroscopy, and mass spectrometry, and purity was established by elemental analysis.

Monoacyl-Homospermines.

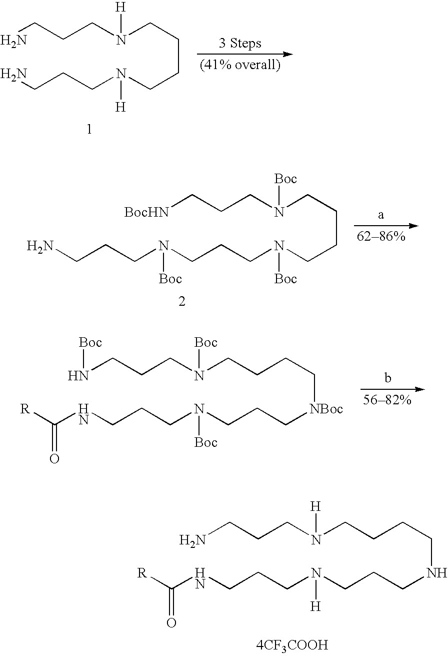

wherein 3a, R=CH₃; 3b, R=C₈H₁₇; 3c, R=C₉H₁₉; 3d, R=C₁₂H₂₅; 3e, R=C₁₄H₂₉; 3f, R=C₁₆H₃₃; 3g, R=C₁₈H₃₇.

wherein 4a, R=CH₃; 4b, R=C₈H₁₇; 4c, R=C₉H₁₉; 4d, R=C₁₂H₂₅; 4e, R=C₁₄H₂₉; 4f, R=C₁₆H₃₃; 4g, R=C₁₈H₃₇.

The reagents were as follows: (a) Ac₂O, py, DMAP, rt. (for 3a), or, RCOCl, DMAP, py, rt. (for 3b-c), or, ROCOCl, EtOAc, aq. NaHCO₃ (for 3d, directly used for the next reaction), or, RCOOH, EDCI, THF, 10 h (for 3e-g); (b) TFA, rt., 8 h.

Bisacyl-Homospermines.

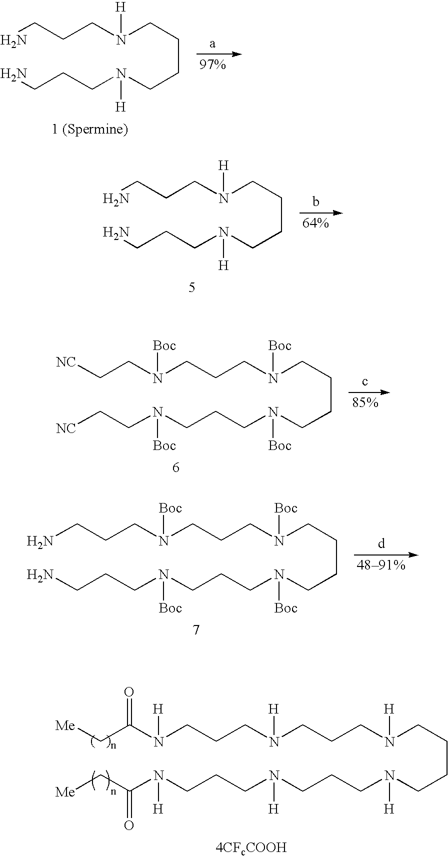

wherein 8a, n=7; 8b, n=8; 8c, n=10; 8d, n=13; 8e,n=15; 8f, n=17.

The reagents were as follows: (a) i. F_{c}CCOOEt (2 eq.), MeOH, −78 to 0° c, 1 h. ii. Boc₂O (excess), O to rt, 1 h. III. Aq. MeOH, NH₃, rt. 25 h.; (b) i. H₂C═CHCN, MeOH, rt., 15 h. II. Boc₂O, CH₂Cl₂, 90 min.; (c) Pd(OH)₂/C, H₂, AcOH, 50 psi. (d) i. RCOOH, EDCI, THF, 10 h. II. TFA, rt, 8 h

### EXAMPLE 2

### Minimum Inhibitory Concentrations

In this example, E. coli strain 9637 and S. aureus strain 13709 were procured from ATCC (Manassas, Va.). For IM permeability assays, E. coli ML-35 (ATCC 43827), a lactose permease-deficient strain with constitutive cytoplasmic β-galactosidase activity was used (26). Calcium chloride transformation of E. coli ML-35 was performed using the plasmid vector pBR322 (6), encoding tetracycline and ampicillin resistance genes (Promega, Madison, Wis.). The transformed strain, E. coli ML-35p, selected by ampicillin resistance, was utilized for the OM permeabilization assay. E. coli ML-35p was maintained on trypticase soy agar plates with 50 μg/ml of ampicillin.

Minimum inhibitory concentrations of the acylpolyamines were determined by broth microdilution method (1) as per NCCLS guidelines. Mid-log phase Mueller-Hinton broth (MHB; non-cation supplemented) cultures of organisms (40 μl; OD₆₀₀ₙₘ adjusted to 0.5 AU, and diluted ten-fold) were added to equal volumes of two-fold serially diluted acylpolyamines in a 384-well microtiter plate with the help of a Biotek Precision 2000 automated microplate pipetting system. The MICs of rifampicin, polymyxin B (PMB), polymyxin B nonapeptide (PMBN), naphthylacetyl spermine trihydrochloride and methoctramine tetrahydrochloride (Sigma, St. Louis, Mo.) were included as reference compounds for comparison of activity. The microtiter plates were sealed and incubated overnight at 37° C. The plates were read at an absorbance of 600 nm. The lowest concentration of an agent inhibiting growth of the organisms was recorded as the MIC.

The polyamine compounds of the present invention showed growth-inhibitory activity against both Gram negative and Gram positive bacteria: The MICs against E. coli ATCC 9637 and S. aureus ATCC 13709 of the acylpolyamines are summarized in Table 1. Also included in Table 1 are the MIC values for naphthylacetylspermine and methoctramine, which are hydrophobically substituted polyamines recently shown to exert membrane-permeabilizing activity; polymyxin B (PMB), a peptide antibiotic known to disrupt OM integrity by binding to LPS; polymyxin B nonapeptide (PMBN), a deacylated derivative of PMB known to effectively permeabilize Gram negative OM, but exerting a highly attenuated antimicrobial potency; and melittin, a cytolytic, highly membrane-active α-helical peptide constituent of bee venom. It is noteworthy that the range of MICs of both the mono-substituted and bis-substituted long-chain aliphatic acylpolyamines used in this study against E. coli is rather narrow (31.25 μM-62.5 μM; two dilutions), while both the mono-(naphthylacetylspermine) and bis-aryl (methoctramine) compounds display significantly lower MICs (1250 μM, and 312.5 μM, respectively; Table 1). The MICs for these latter two compounds reported by Yasuda et al., Mode of action of novel polyamines increasing the permeability of bacterial outer membrane, Int. J. Antimicrob. Agents 24:67-71 (2004) against E. coli W3110 were >267 μM (>128 mg/l), and 22 μM (16 mg/l), respectively. These discrepancies may be attributable to the differences in the strains used.

TABLE 1
MICs of lipopolyamines against E. coil and S. aureus

| | MIC | |
|---|---|---|
| | μM; (μg/ml) | |
| Compound | E. coli ATCC 9637 | S. aureus ATCC 13709 |
| 4a | 62.5; (47.3) | 250; (189.3) |
| 4b | 62.5; (53.4) | 125; (106.96) |
| 4c | 62.5; (54.3) | 62.5; (54.3) |
| 4d | 31.25; (28.9) | 15.6; (14.4) |
| 4e | 31.25; (29.3) | 15.6; (14.6) |
| 4f | 62.5; (60.4) | 15.6; (15.1) |
| 4g | 62.5; (62.2) | 15.6; (15.5) |
| 8a | 31.25; (32.9) | 15.6; (16.4) |
| 8b | 31.25; (33.7) | 3.9; (4.2) |
| 8c | 31.25; (35.5) | 15.6; (17.7) |
| 8d | 62.5; (76.3) | 250; (305.3) |
| 8e | 62.5; (79.4) | 250; (319.3) |
| 8f | 62.5; (83.3) | 125; (166.7) |
| PMB | 3.9; (5.4) | 125; (173.2) |
| PMBN | 250; (240.7) | 500; (481.5) |
| Melittin | 175; (498.1) | 5.5; (15.6) |
| Naphthylacetylspermine | 1250; (599.8) | 1250; (599.8) |
| Methoctramine | 312.5; (227.7) | 156.25; (113.8) |

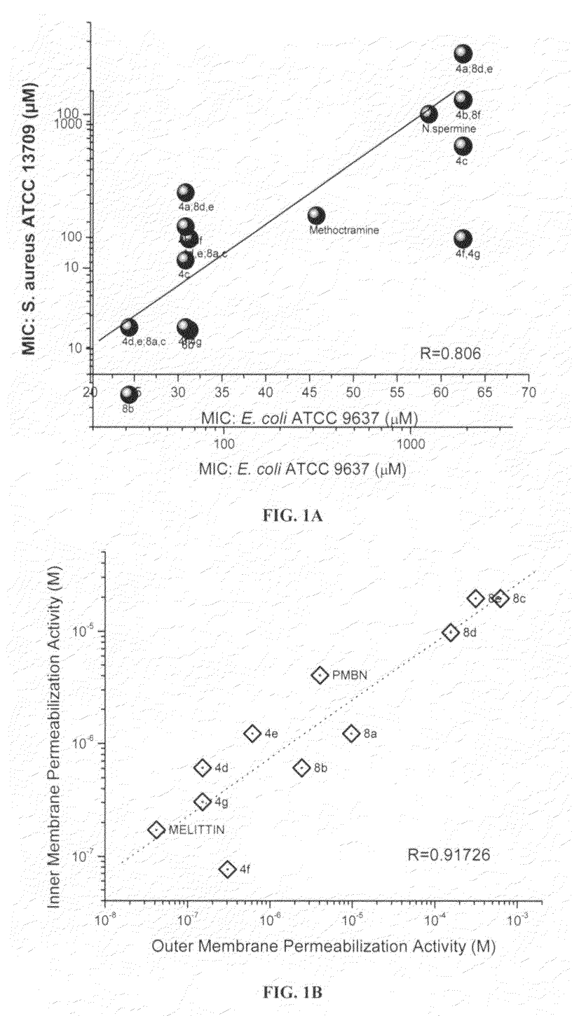

A much wider dispersion in intrinsic antibacterial effect against S. aureus is observed, with a range of 4 μM (8b) to 250 μM (4a). A cursory inspection of the data would suggest that the antimicrobial potency against S. aureus could be correlated with the hydrophobicity of the acyl group. For instance, 8b, a bis-C₈-acyl compound is expected to be more surface active (see below) than the monoacetyl 4a derivative, which would be consonant with reports in the literature indicating that Gram positive organisms are, in general, more susceptible to cationic amphipathic substances, with susceptibility increasing with amphipathicity (15, 18, 45). However, the MIC against S. aureus of 8a and 8c, both immediate structural neighbors of 8b, are considerably higher. Furthermore, we observed a poor correlation between the MICs against E. coli and S. aureus (FIG. 1A). These results point to the potential complexity of physicochemical features that dictate structure-activity relationships, which may not be directly attributable to hydrophobicity. Indeed, measures of hydrophobicity such as C₁₈ reverse-phase HPLC retention times, or computed logP values (data not shown), or surface tension measurements (see below) correlate poorly with the observed antibacterial activities.

### EXAMPLE 3

### Outer and Inner Membrane Permeability

In this example, the mechanisms and structure-activity relationships underlying the membrane permeabilizing actions of the compounds were investigated. Such properties provide the possibility of employing such compounds as adjuncts to conventional chemotherapy against resistant organisms, for purposes of sequestering endotoxin released as a consequence of Gram negative bacterial lysis. Thus, in this example, it was first investigated if the acylpolyamines would act on both the IM and the OM, presumably as a consequence of nonspecific membranophilic effects as has reported for a variety of cationic amphipathic peptides such as melittin, defensins, and bactenecins or, selectively perturb the OM in the manner of PMB.

The OM permeability was measured using a procedure similar to that reported by Lehrer et al., Concurrent assessment of inner and outer membrane permeabilization and bacteriolysis in E. coli by multiple-wavelength spectrophotometry, J. Immunol. Methods 108:153-158 (1988), which was modified for high-throughput read-out. Nitrocefin (Calbiochem, San Diego, Calif.) was used for the determination of periplasmic β-lactamase activity since PADAC has been reported to be frequently insensitive to β-lactamase activity in clinically relevant strains of Staphylococcus. See Anhalt et al., Failure of Padac test strips to detect staphylococcal β-lactamase, Antimicrob. Agents Chemother. 21:993-994 (1982). Harvested mid-log phase cultures of E. coli ML-35p (OD₆₀₀ₙₘ adjusted to 0.5 AU) grown in trypticase soy broth were washed thrice with normal saline (0.9%). Nitrocefin was added to a final concentration of 50 μg/ml to the washed bacterial suspension, which was then added to the serially diluted compounds in a 384-well microtiter plate as described earlier. PMB, PMBN and melittin (Sigma, St. Louis, Mo.), a potent membrane active bee-venom peptide were used as reference compounds. After various times of incubation at 37° C., β-lactamase activity was measured spectrophotometrically at 486 nm using an automated Spectramax M2 instrument (Molecular Devices, Sunnyvale, Calif.).

The IM permeability was measured using o-nitrophenyl-β-D-galactopyranoside (“ONPG”; Sigma, St. Louis, Mo.) as the substrate to determine the β-galactosidase activity. See Lehrer et al., Concurrent assessment of inner and outer membrane permeabilization and bacteriolysis in E. coli by multiple-wavelength spectrophotometry, J. Immunol. Methods 108:153-158 (1988). Washed cultures of E coli ML-35 mixed with 1.5 mM of ONPG in normal saline (0.9%) were added to serially diluted compounds in a 384-well microtiter plate. PMBN and melittin were used as the controls. The production of o-nitrophenol was quantified absorptimetrically at 420 nm after an incubation period of 1 hour at 37° C.

OM and IM permeability were determined respectively from dose-response curves of nitrocefin and ONPG hydrolysis rates as described in Lehrer et al., Concurrent assessment of inner and outer membrane permeabilization and bacteriolysis in E. coli by multiple-wavelength spectrophotometry, J. Immunol. Methods 108:153-158 (1988). As shown in FIG. 1B, a direct linear relationship was observed between OM and IM permeabilizing activities. Furthermore, these two events seem tightly coupled with near-identical kinetics even under conditions of high osmotic strength (data not shown) as has also been observed with antibacterial host-defense peptides. See Lehrer et al., Interaction of human defensins with Escherichia coli. Mechanism of bactericidal activity, J. Clin. Invest. 84:553-561 (1989). Although IM damage would necessarily require antecedent OM lysis, the lag-times in the hydrolysis of the chromogenic substrates are too short for a clear discrimination to be observed under the experimental conditions employed. As has been hypothesized in the case of antibacterial peptides, the mechanism of bacterial killing likely involves loss of IM integrity.

### EXAMPLE 4

### Effect of Acylamines on MIC of Rifampin

Perturbation of the outer membrane permeability barrier greatly sensitizes Gram negative organisms to otherwise impermeable hydrophobic solutes, rifampicin being a classic example To determine the effect of acylpolyamines on the MIC of hydrophobic antibiotic, rifampicin, E. coli strain 9637 was used. Overnight cultures of E. coli grown in MHB preincubated with 10 μM of acylpolyamines were added to serially diluted rifampicin in a 384-well microtiter plate. After incubation at 37° C. overnight, the MIC was determined. Controls included PMBN and melittin (positive) and rifampicin alone (negative). All experiments were run in triplicate. Table 2 shows the results of the tested compounds.

| | Sensitization to |
|---|---|
| Compound | Rifampin |
| 4a | 31.25 |
| 4b | 7.81 |
| 4c | 7.81 |
| 4d | 62.5 |
| 4e | 7.81 |
| 4f | 15.6 |
| 8a | 0.00381 |
| 8b | 0.00381 |
| 8c | 3.9 |
| 8d | 15.6 |
| 8e | 15.6 |
| 8f | 15.6 |
| PMB | 0.2 |

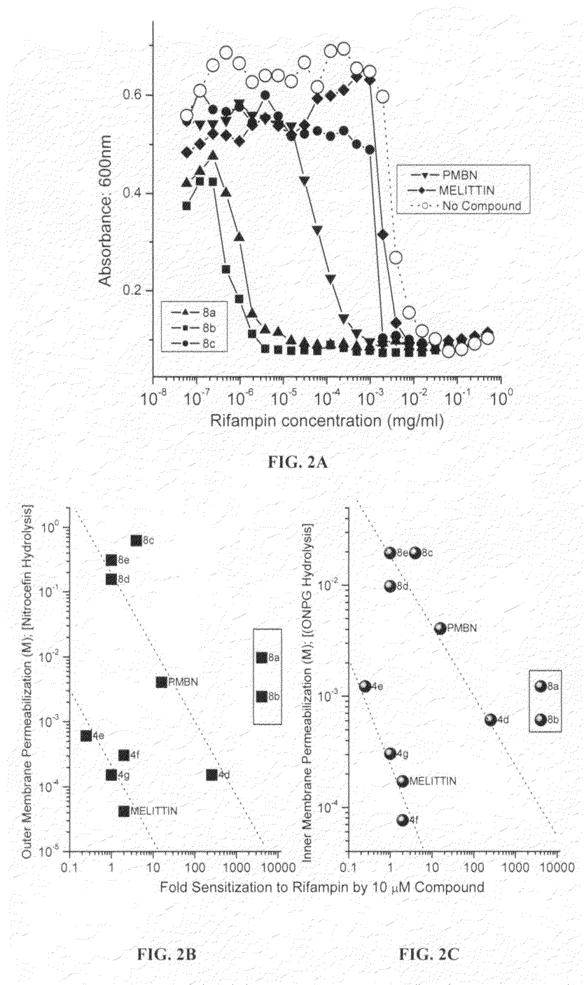

The results are also presented in FIG. 2. A simple linear relationship between both OM and IM permeabilization and sensitization to rifampicin was expected. Surprisingly, a clear demarcation of the compounds into distinct subsets with differential activity was observed (FIGS. 2B and 2C1). Compound 4e-g as well as melittin, all of which are potently membrane-permeabilizing, sensitized E. coli ATCC 9637 to an apparently lesser degree than 8c-e, PMBN, and 4d. In both these groups of compounds, there was indeed a demonstrable direct correlation between permeabilizing activity and rifampicin sensitization (FIGS. 2B and 2C). Distinct from these two groups, however, 8a and 8b were found to possess extremely high sensitizing activity. 10 μM of PMBN, a prototype membrane-permeabilizing compound, lowered the MIC of rifampicin from 15.625 μg/ml to 0.976 μg/ml (16-fold), while the sensitization activity of 8a and 8b were 4096-fold (FIGS. 2B and 2C). In light of the fact that the nitrocefin and ONPG hydrolysis assays do not discriminate between complete membrane lysis of a small fraction of bacteria from partial lysis of a larger fraction of bacteria, these results may be interpreted as follows: long-chain mono-acyl compounds such as 4g, 4f and melittin are highly membrane-active (FIG. 1, bottom panel), and are likely to lyse immediately the fraction of the organisms that the compound first comes in contact with, given their higher propensity to self-aggregate in membranes; the remainder of the bacteria are unaffected, continue to proliferate in culture, and thus do not manifest in an apparent enhancement of susceptibility to rifampicin. In contrast, the fact that the bis-acyl compounds are less hemolytic than the mono-acyl analogues (see below) suggest that the bis-acyl 8a and 8b compounds may interact with outer membranes more diffusely. The resultant non-lethal perturbation of a greater fraction of the bacteria is likely reflected as a profound enhancement in the susceptibility to hydrophobic antibiotics such as rifampicin. While the definitive interpretation of these results must await detailed experiments involving measurements with the fraction of bacteria with depolarized membrane potentials using a method such as flow-cytometry, the highly pronounced sensitizing activity of compounds such as 8a and 8b, relative to PMBN is particularly noteworthy.

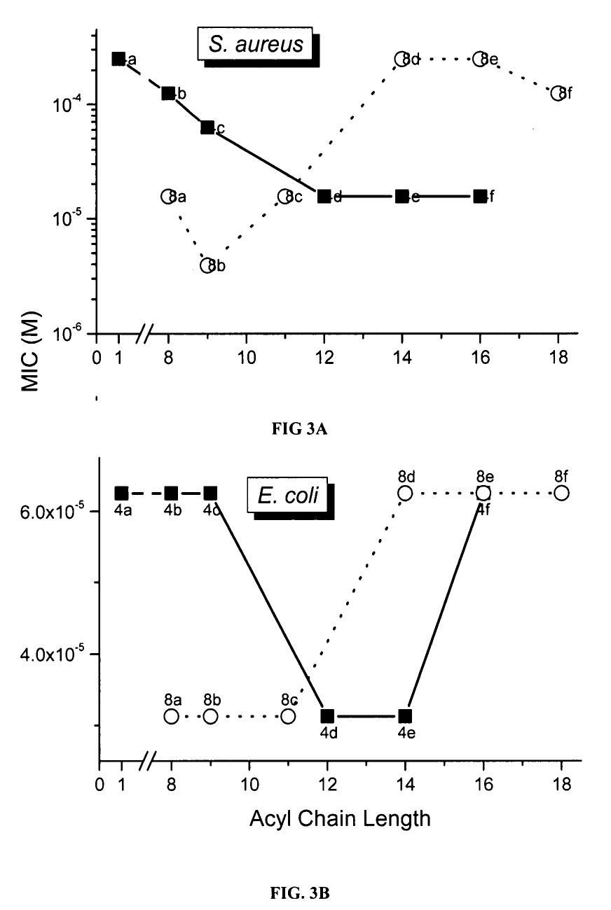

It was previously shown that the carbon number (hydrophobicity) of the homologous series of mono- and bis-acyl polyamines was a critical structural determinant of LPS-neutralizing potency. In particular, for the mono-acyl 4 series of compounds, there was a progressive increase in LPS-neutralizing potency, while for the bis-acyl 8 series, the activity progressively decreased with acyl chains longer than dodecyl (C₁₂). Thus, this example also examined possible correlations with MICs against E. coli and to verify if the activity profile would be similar in inhibiting the growth of S. aureus. The results shown in FIG. 3 indicate that against both organisms, a very similar structure-activity correlation is observed. Thus, for the 4 compounds, acyl chain lengths from C₁₂ to C₁₆ result in maximal antimicrobial efficacy against S. aureus. Maximal antibacterial effects are observed between C₁₂ to C₁₄ against E. coli, with the activity falling off at C₁₆, suggesting that the structural requisites for optimal interaction with the Gram negative outer membrane are rather specific. For the 8 series, however, the converse is true with the short chain (C₈₋₁₁) analogues exhibiting maximal antibacterial effect (FIG. 3); the decline in activity in the higher homologues in the 8 series is ascribable to progressive loss of aqueous solubility as reported earlier (30). It is of interest that a very similar structure-activity relationship was observed with these compounds in terms of inhibition of LPS-induced TNF-α and nitric oxide production in murine macrophages (30). The similarities of the 4 and 8 series between antimicrobial activity against Gram negative bacteria on the one hand, and sequestration of LPS on the other, would suggest that the antimicrobial activity may be mediated via the interaction of these compounds with the outer membrane.

### EXAMPLE 5

### Surface Tension Measurements

Charged, amphipathic molecules are surface-active, and can be cytolytic to mammalian cells. In this example, the surface activity of the test compounds was measured via dynamic bubble pressure and surface age tensiometry (Fainerman et al., Maximum bubble pressure tensiometry—an analysis of experimental constraints, Adv. Colloids Interface Sci. 108-109:287-301 (2004)) using a Krüss PocketDyne instrument (Krüss GmbH, Hamburg, Germany) as described earlier in Miller et al., Lipopolysaccharide Sequestrants. Structural Correlates of Activity and Toxicity in Novel Acylhomospermines, J. Med. Chem. 48:2589-2599 (2005). Samples were at 500 μM concentration in 50 mM Tris buffer, pH 7.4 containing 5% DMSO. The instrument was calibrated with water at 25° C. (72 mN/m) and surface tension values were recorded over a range of bubble surface ages from 100 to 1500 ms at 25° C.

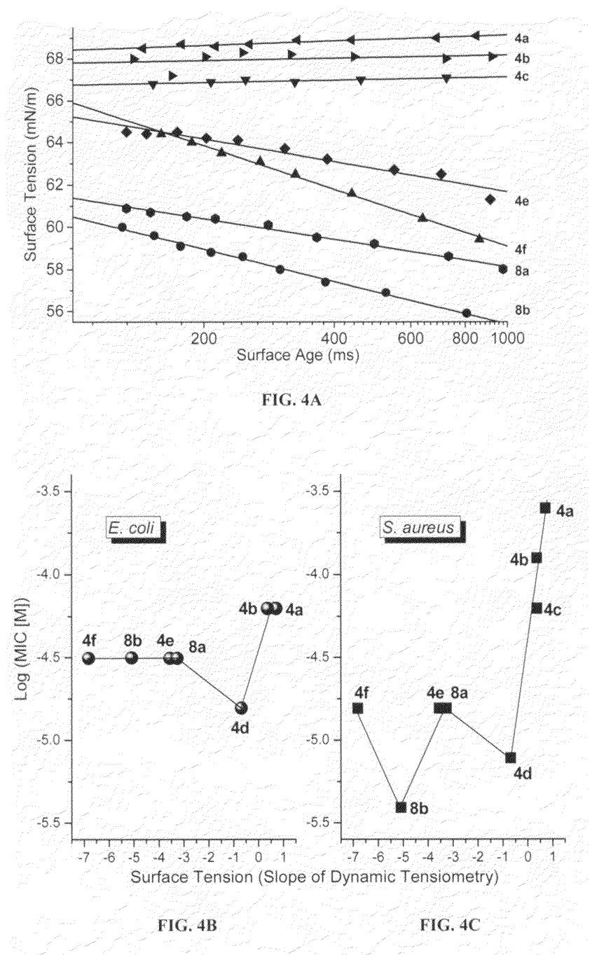

The 8 series are analogous to “Gemini surfactants,” so named after their twin-headed structures and could, possibly, display nonspecific cytotoxicity because of membrane-perturbing activity. As expected, the ‘Gemini’-like 8a and 8b (measured in 5% DMSO to ensure solubility; the higher homologs were insoluble and could not be tested), are indeed considerably surface active (FIG. 4). For the 4 series (all of which were freely soluble in 5% DMSO), there is a distinct correlation between acyl chain length and surface tension-lowering activity, as could be expected, with homologs with longer acyl chains becoming progressively more surface active (FIG. 4A). Unexpectedly, there was a lack of correlation between surface activity and MIC against both E. coli and S. aureus, with 4d (for both organisms) and 8b (for S. aureus) being significant outliers (FIGS. 4B and 4C). These results suggest a specific interaction of these two compounds with bacterial membranes, rather than a non-specific, surface activity-related membrane perturbation. Raman spectroscopic experiments are being planned which may provide a better understanding of the mechanisms of interfacial phenomena at the bacterial cell surface.

### EXAMPLE 6

### Hemolytic Activity

In order to test the hypothesis that the antibacterial activities of the acylpolyamines are a consequence of membrane-permeabilization due to their cationic amphipathic nature, this example sought to correlate surface activity of these compounds with hemolytic potency. See Ross et al., Micellar aggregation and membrane partitioning of bile salts, fatty acids, sodium dodecyl sulfate, and sugar-conjugated fatty acids: correlation with hemolytic potency and implications for drug delivery, Mol. Pharmaceutics. 1:233-245 (2004). Erythrocyte damage was measured using two different techniques. In the first, hemolysis was quantified using extremely diluted, aged human whole blood such that the effects of the compounds binding to plasma proteins would be negligible, and the hemolytic activity would be magnified because of increased osmotic fragility of the erythrocytes as a consequence of depleted Na⁺ K⁺ ATPase activity. See Nagini et al., Biochemical indicators of membrane damage in the plasma and erythrocytes of rats fed the peroxisome proliferator di(2-ethylhexyl)phthalate, Med. Sci. Res. 25:119-121 (1997). Dilute erythrocyte suspensions were prepared by diluting one-week-old whole blood obtained by venipuncture from healthy human volunteers 1:1000 in isotonic (0.9 g/100 ml) saline solution to which was added graded doses of compound. Absorptimetric determinations of hemoglobin released from such dilute erythrocyte suspensions were not reliable. The samples were therefore examined with a Beckman-Coulter Vi-Cell™ Cell Viability Analyzer (Beckman-Coulter, Hialeah, Fla.). This instrument implements an automated intravital trypan blue exclusion method using real-time automated video microscopy. Measurement parameters for erythrocytes were gated appropriately on control erythrocytes to specify thresholds of cell recognition and viability. Data on total number of cells/ml and viable cells/ml were collected through 50 captured images per sample with a counting accuracy of ±3%. In order to examine the effect of plasma proteins on the surface activity, some of the experiments were repeated in the presence of near-physiological concentrations of human serum albumin. Because it became apparent that the compounds were binding strongly to albumin, thereby resulting in an almost complete abrogation of hemolytic activity, it was of interest to examine the compounds under physiological conditions. The second method, consequently, was designed to examine the effects of the compounds on whole blood. 100 μl of serially diluted compounds were mixed with an equal volume of fresh, undiluted, EDTA-anticoagulated human blood in a 96-well microplate using an automated liquid handler. After incubation at 37° C. for 30 min, the plates were centrifuged at 3000 RPM for 10 min, 80 μl of supernatants transferred to a fresh plate, and the amount of free hemoglobin released into the supernatant was quantified using absorptimetry at 570 nm. In the latter assay, melittin, a potently hemolytic α-helical bee venom peptide was used as positive control. See David et al., Interaction of melittin with endotoxic lipid A, Biochim. Biophys. Acta 1123:269-274 (1992).

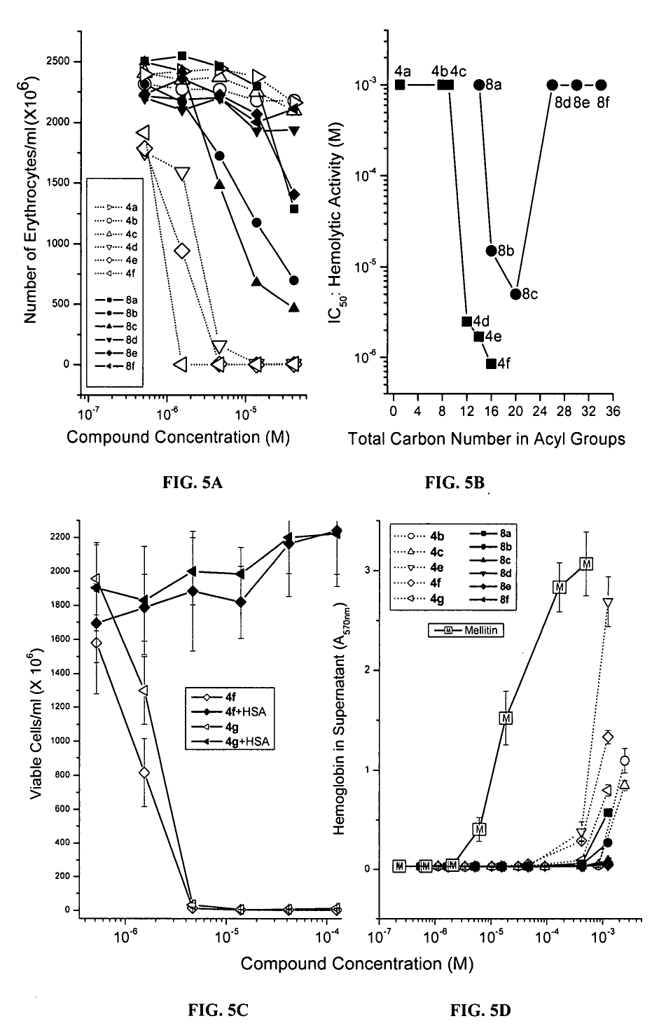

The hemolysis induced by the compounds was quantified using an extremely dilute suspension of washed, aged human erythrocytes under protein-free conditions (isotonic saline). In this assay, erythrocytes become exquisitely susceptible to membrane damage and lysis, not only because of increased osmotic fragility of the erythrocytes due to depleted Na⁺ K⁺ ATPase activity, but also due to the absence of ‘buffering’ effects of plasma proteins. Increasing acyl chain lengths is paralleled by higher hemolytic activity, particularly for the 4 series (FIG. 5A). It is to be noted that the hemolytic activity of the bis-acyl 8 compounds is biphasic, increasing substantially from 8a (C₇) to 8c (C₁₀), and then diminishing at higher carbon chain lengths (FIG. 5B) due to decreasing solubility. Thus, for the 8 series, the lack of adequate aqueous solubility may likely account for the progressive decline in antimicrobial activity of the higher homologs as shown in FIG. 3.

The pronounced hemolytic activity of long-chain acylated compounds such as 4e and 4f (100% hemolysis at 1-5 μM) occasioned concern, and we questioned if the results of this assay employing deliberately exaggerated erythrocytic fragility would be physiologically relevant; that is, if these compounds would likely cause intravascular hemolysis in vivo if administered parenterally. In the course of our investigations, we found that the acylpolyamines bind strongly to albumin. Detailed biophysical studies on the characterization of the binding site on albumin, stoichiometry, and dependence of binding affinity on mono-versus bis-acylation and acyl chain length will be published elsewhere. The hemolytic activity of the acylpolyamines is completely abrogated, even at very high concentrations, in the presence of physiological concentrations (˜650 μM) of human serum albumin as observed with 4f and 4g, shown as representative data (FIG. 5C), indicating that a large fraction of these compounds is bound to albumin and that the protein-bound form would be unlikely to exert toxicity in vivo. The hemolytic activities of these compounds were therefore reexamined using human whole blood and, consistent with our hypothesis, significant hemolysis was observed starting to occur only at millimolar concentrations (FIG. 5D). In these latter experiments, melittin, an α-helical 26-residue hemolytic bee venom peptide (3, 9), caused hemolysis at low micromolar concentrations. Furthermore, preliminary acute (up to 1 mg/mouse; one dose subcutaneously) and subacute (100 μg/mouse, subcutaneously, for 15 days) toxicity studies in CF-1 mice with 4d and 8a have not revealed any detectable toxicity.

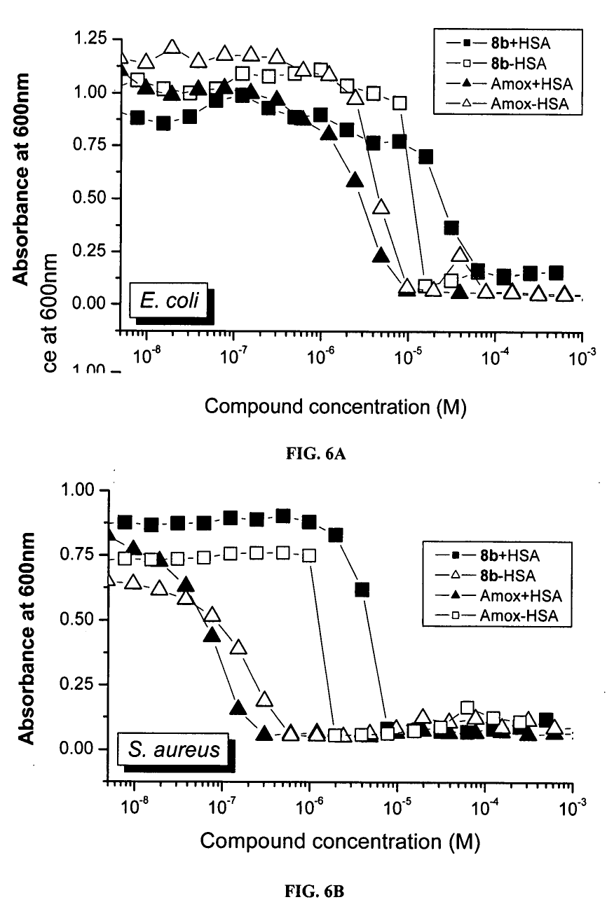

The strong binding of the acylpolyamines to human serum albumin raised the question whether the antimicrobial effects of these compounds would be completely abrogated in the presence of physiological concentrations of albumin. In a preliminary experiment, the antimicrobial effect of 8b, chosen as a representative compound, was examined in the presence, or absence of physiological concentrations of human serum albumin (4.5 g/100 ml; 677 μM). As shown in FIG. 6, there is approximately a four-fold attenuation of MIC values (E. coli: 31.25 μM to 125 μM; S. aureus: 3.9 μM to 15.6 μM). These data clearly demonstrate that the acylpolyamines retain significant antibacterial activity in the presence of albumin, suggesting that these compounds may also be active in vivo. Indeed, the LPS-sequestration properties of these compounds are also virtually unchanged in the presence of albumin. These results, taken together, would suggest that the K_{off} rate of the lipopolyamine:albumin complex is rather fast, affording concentrations of free lipopolyamine capable of sequestering monomeric LPS or interacting with bacterial membranes, and yet considerably lower than the CMC value, which would account for the lack of hemolytic/membrane-active properties under these conditions. Surface plasmon resonance experiments are currently underway to test this hypothesis.

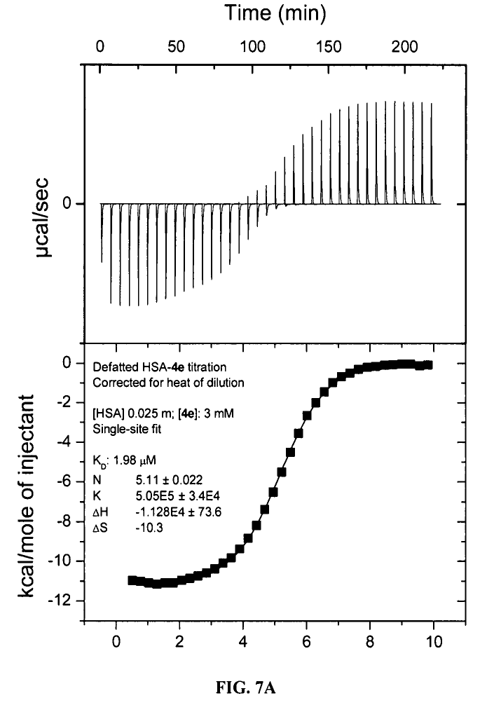

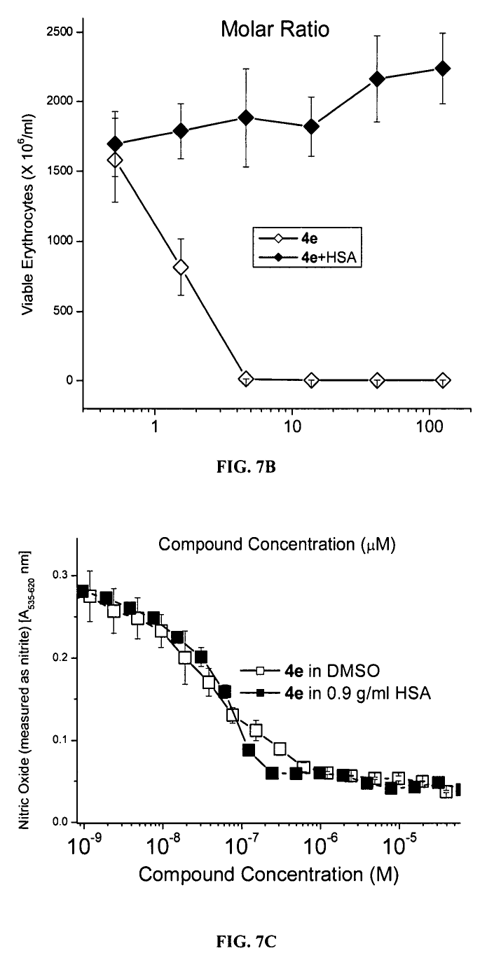

In a separate experiment, it was determined that compounds such as 4e bind HSA with a K_{D} (determined by isothermal titration calorimetry) of 1.98 μM, and a ligand:protein stoichiometry of 5:1 (FIG. 7A). The binding sites on HSA were determined to be the multiple fatty acid binding pockets because of specific displacement of dansylsarcosine and cis-parinaric acid, but not warfarin by the lipopolyamines. The consequence of the interaction is a complete abrogation of the surface-activity (and hemolytic activity) of these cationic amphipathic compounds (FIG. 7B). Gratifyingly, the complexation of the lipopolyamines with HSA results in no change in potency in a variety of in vitro assays (FIG. 7C) as well as in the murine model of endotoxic shock, implying that either the K_{off} rate of the lipopolyamine:HSA complex is very rapid, or that compounds such as 4e bind to and sequester LPS as a ternary complex with albumin, as has been observed in the case of polymyxin B. These fortuitous observations have led to a formulation compatible with systemic administration of lipopolyamines.

The compounds of the present invention may be readily solubilized in isotonic saline containing physiological concentrations of albumin. Dose-response profiles are identical to that obtained with DMSO solutions with the advantage that repeated intravenous or intraperitoneal injections result in no observable thrombophlebitis or sterile peritonitis in mice.

### EXAMPLE 7

### Acute and Subacute Toxicity Studies in Mice

In acute toxicity studies, graded doses of Compound 4e (100 μg-500 μg) in 0.2 ml saline was injected intraperitoneally or subcutaneously in cohorts of 5 CF-1 outbred mice per dose, according to IACUC-approved protocols. Clinical signs of acute toxicity were monitored for 48 h following a single injection. In subacute studies, 100 μg of 4e was injected daily in the subcutaneous tissue of the flank region, the sites being alternated every day, for a duration of 15 days. The animals were monitored daily for signs of local irritation, weight loss, and food consumption.

### EXAMPLE 8

### Synthesis of Alkyl Polyamines (DS-96)

In this example, a compound NH₂C₃H₆NHC₄H₈NHC₃H₆NHC₃H₆NHC₁₆H₃₃ (DS-96) was prepared according to the scheme below:

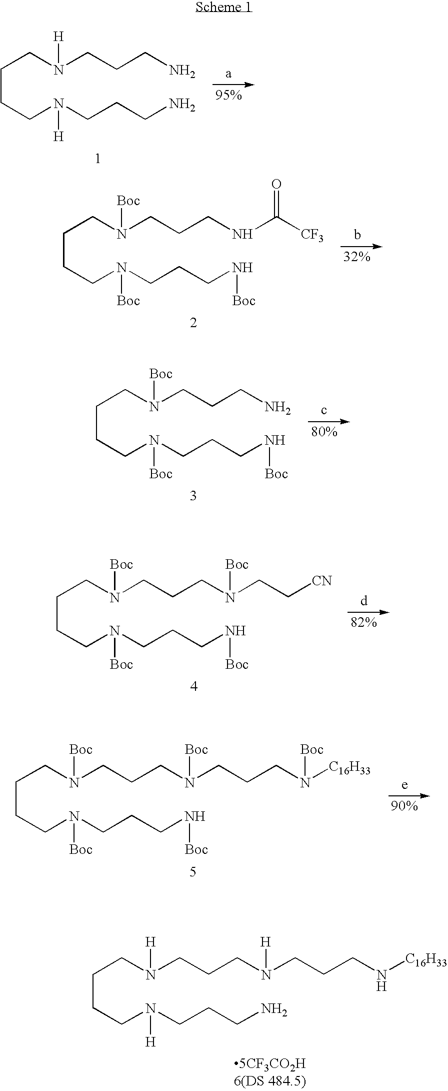

wherein a. (i) CF₃COOEt, MeOH, −78° C. to 0° C., 1 h; (ii) Boc₂O (excess), 0° C. to rt, 12 h. b. NaBH₄, MeOH, reflux 60° C. c. (i) CH₂═CHCN, MeOH, r.t, 15 h; (ii) Boc₂O (excess), MeOH, rt, 12 h. d. (i) C₁₆H₃₃NH₂ (excess), Pd(OH)₂/C, H₂, 60 psi; (ii) Boc₂O (excess), MeOH, rt, 12 h. e. CF₃CO₂H (excess), rt.

Chemistry. All of the solvents and reagents used were obtained commercially and used as such unless noted otherwise. Moisture or air-sensitive reactions were conducted under argon atmosphere in oven dried (120° C.) glass apparatus. Solvents were removed under reduced pressure using standard rotary evaporators. Flash Column chromatography was carried out using silica gel 635 (60-100 mesh) while, thin layer chromatography was carried out on silica gel CCM precoated aluminum sheets. All yields reported refer to isolated material judged to be homogenous by TLC, NMR spectroscopy and Mass spectroscopy. NMR spectra were recorded with the chemical shifts (6) reported in ppm to Me₄Si (for ¹H) and CDCl₃ (for ¹³C) or DMSO-d₆ (for ¹³C) as internal standards respectively.

The tri-Boc-trifluoroacetate-polyamine 2 and tri-Boc-spermine 3 were synthesized using the procedures from Example 1, while compound 5 was synthesized using Sajiki H.; Ikawa T.; Hirota K. Reductive and Catalytic Monoalkylation of Primary amines Using Nitriles as an Alkylating reagent Org. Lett. 2004, 26, 4977-4980

Synthesis of Compound 2. To a solution of spermine 1 (2 g, 9.9 mmol) in methanol (50 mL) at −78° C. was added dropwise ethyl trifluoroacetate (1.17 ml, 9.9 mmol) over 30 min and the solution was stirred for another 30 minutes. The temperature was raised to 0° C. and an excess of di-tert-butyl dicarbonate (21.6 g, 99.0 mmol) in methanol (10 ml) was added over 10 minutes. The reaction was then warmed to 25° C. for 15 hours. After removal of solvent under vacuum, the residue was purified by flash column chromatography (Hexane/EtOAc=1:1) to afford the title compound 2 as a white solid (5.71 g, 95%): proton data—paper.

Synthesis of Compound 3.

To a solution of compound 2 (5.71 g, 9.5 mmol) in absolute ethanol (50 mL) was added sodium borohydride (1.05 g, 28.6 mmol) and the mixture was refluxed at 60° C. for 12 hours. After removal of solvent, the residue was taken up in CH₂Cl₂ and washed sequentially with water (25 mL×3). The organic layer was dried over Na₂SO₄, filtered and solvent was evaporated under reduced pressure to give compound 3 (1.52 g, 32%) as viscous oil: proton data—paper.

Synthesis of Compound 4.

To a solution of compound 3 (0.8 g, 1.59 mmol) in methanol (30 mL) was added acrylonitrile (0.105 mL, 1.59 mmol) and the mixture stirred at room temperature for 15 hours. After removal of solvent under high vaccum, the crude mono-nitrile derivative was dissolved in 30 ml of CH₂Cl₂ followed by addition of a solution of di-tert-butyl dicarbonate (1.74 g, 7.9 mmol). The resulting solution was stirred for 12 hours at room temperature, concentrated in vaccum, and purified by flash column chromatography (Hexane/EtOAc=3:1) to give colorless oil 4 (0.83 g, 80%). ¹H NMR (400 MHz, CDCl₃) δ 1.4 (s, 36H), 1.55 (s, 4H), 1.81 (m, 4H), 2.67 (br s, 2H), 2.96-3.0 (br m, 12H), 3.3 (t, 2H); ¹³C NMR (100.6 MHz, CDCl₃); 16.9, 17.4, 23.5, 24.6 25.8, 28.3, 36.6, 37.4, 43.5, 43.9, 44.5, 45.4, 46.5, 46.7, 78.8, 79.4, 80.5, 118.2, 155.1, 155.4, 156.0; MS(ESI) calculated for C₃₃H₆₁N₅O₈ m/z 655.4 found 678.4 (MNa)⁺.

Synthesis of Compound 5.

A solution of mono-nitrile 4 (0.35 g, 0.53 mmol) and hexadecylamine (0.63 g, 2.94 mmol) in methanol (20 ml) was hydrogenated over Pd(OH)₂/C (0.3 g) at 60 psi pressure for 12 hours. The catalyst was removed by filtration and the residue was washed thoroughly with methanol. After removal of solvent under high vaccum, the crude secondary amine alkylated compound (0.41 g, 0.46 mmol, 87%) was dissolved in methanol (30 ml) followed by the addition of a solution of di-tert-butyl dicarbonate (1.01 g, 1.85 mmol). The resulting solution was stirred for 12 h at ambient temperature, concentrated in vaccum and purified by flash column chromatography (Hexanes/EtOAc=4:1) to give compound 5 (0.190 g, 40%) as a viscous oil. ¹H NMR (400 MHz, CDCl₃) δ 0.86 (t, J=11.7, 3H), 1.26-1.29 (s, 26H), 1.42-1.52 (br s, 49H), 1.81 (br m, 6H), 3.1 (br s, 18H); ¹³C NMR (100.6 MHz, CDCl₃); 9.4, 14.0, 22.6, 23.4, 24.6, 26.8, 27.5, 28.0, 28.3, 29.3, 29.6, 31.8, 33.7, 36.6, 37.2, 43.6, 44.7, 46.3, 46.7, 47.0, 76.7, 77.0, 77.4, 78.8, 79.0, 79.2, 91.6, 155.3, 155.4, 156.0; MS(ESI) calculated for C₅₄H₁₀₅N₅O₁₀ m/z 983.7 found 1006.7 (MNa)⁺.

Synthesis of Compound 6.

The resulting Boc-protected mono-acylated polyamine was dissolved in excess (25 mL) of dry trifluoroacetic acid and stirred at room temperature for 8 hours. Excess solvent was removed by purging nitrogen and the residue was thoroughly washed with diethyl ether to obtain white flaky solid 6 (0.170 g, 90%). ¹H NMR (400 MHz, DMSO-d₆) δ 0.85 (t, J=6.6 Hz, 3H), 1.25 (br s, 26H), 1.41 (br s, 6H) 1.55-1.65 (br s, 6H), 2.88 (br m, 18H); ¹³C NMR (100.6 MHz, DMSO-d₆) 22.8, 23.0, 24.2, 25.8, 26.3, 28.9, 29.2, 29.3, 29.4, 31.7, 36.6, 39.5, 39.7, 40.1, 40.4, 40.6, 44.3, 44.5, 46.5, 47.2, 159.0, 159.3; MS(ESI) calculated for C₂₉H₆₅N₅ (free amine) m/z 483.4 found 484.4 (MH)⁺.

### EXAMPLE 9

### Synthesis of EVK-203

In this example, EVK-203 was prepared. Following a literature procedure of Haldar et al., Incorporation of Multiple Head Groups Leads to Impressive Antibacterial Activity. J. Med. Chem. 48:3823-3831 (2005), hexadecanal (1) was prepared by oxidation of commercially available 1-hexadecanol, while the tetra-Boc-polyamine (3) was synthesized following procedure from Example 1.

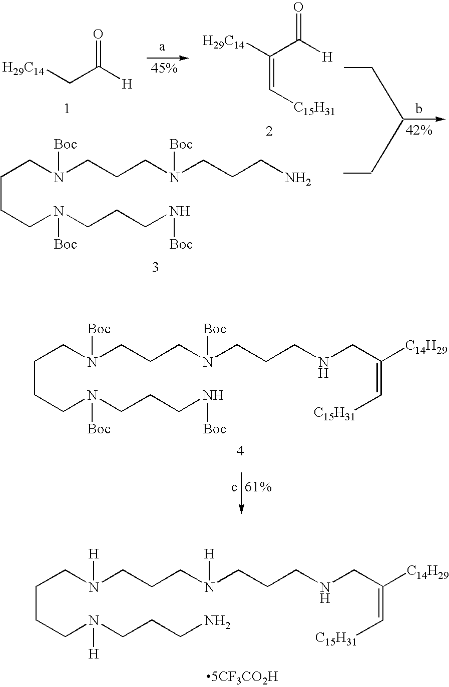

To prepare compound 2, a mixture of the aldehyde 1 (1.2 g, 5 mmol) and anhydrous AlCl₃ (2.6 g, 20 mmol) in pyridine (80 mL) was refluxed for 30 minutes. After cooling to room temperature, the reaction mixture was diluted with diethyl ether (250 mL), the precipitated solid was removed by filtration and the filtrate concentrated under reduced pressure. The residue was purified by flash column chromatography (Hexane/EtOAc=99/1) to yield the product 2 as a low melting solid (1.05 g, 45%). ¹H NMR (400 MHz, CDCl₃) δ 0.90 (t, J=7.04 Hz, 6H), 1.28 (br s, 48H), 1.47-1.55 (m, 2H), 2.22-2.31 (m, 2H), 2.34-2.39 (m, 2H), 6.45 (t J=7.44 Hz, 1H), 9.35 (s, 1H); MS (ESI) calcd for C₃₂H₆₂O m/z 462.4, found 463.5 (MH)⁺.

To prepare compound 4, to a solution of the aldehyde 1 (0.2 g, 0.3 mmol) anhydrous methanol (8 mL) at room temperature were added anhydrous MgSO₄ (ca. Ig) and a solution of the aldehyde 2 (0.2 g, 0.43 mmol) in THF (4 mL). The resulting mixture was stirred at room temperature overnight, followed by addition of NaBH₄ (60 mg, 1.6 mmol). After stirring for another 4 hours, the reaction mixture was concentrated under reduced pressure, the residue was dissolved in CHCl₃ (50 mL) and washed with water (2×5 mL). The organic layer was dried over Na₂SO₄, solvent removed under vacuum and the residue purified by flash column chromatography (CH₂Cl₂:MeOH:NH₄OH) (95:5:1) affording the product 4 (0.14 g, 42%) as an oily liquid. ¹H NMR (400 MHz, CDCl₃) δ 0.88 (t, J=6.6 Hz, 6H), 1.27 (br s, 50H), 1.46 (s, 38H), 1.70-1.74 (br m, 6H), 2.02-2.14 (br m, 6H), 2.71(br s, 2H), 3.05-3.35 (br m, 16H), 5.29 (br t, J=6.68 Hz, 1H); ¹³C NMR (100.6 MHz, CDCl₃); 14.0, 22.6, 25.9, 27.5, 28.3, 28.4, 28.5, 28.8, 29.2, 29.4, 29.5, 29.6, 29.9, 31.8, 31.9, 37.2, 43.8, 44.1, 44.6, 46.4, 46.7, 55.3, 78.8, 79.2, 126.3, 137.5, 155.3, 155.4, 156.0; MS(ESI) calcd for C₆₅H₁₂₇N₅O₈ m/z 1106.7 found 1107.0 (MH)⁺.

To prepare compound 5 (EVK-203), removal of the Boc-protecting groups were carried out by treatment of compound 4 (50 mg, 0.045 mmol) with an excess of trifluoroacetic acid (6 mL) and stirring the solution at room temperature overnight. Concentration of the reaction mixture under reduced pressure, followed by trituration of the residual liquid with methylene chloride provide the product 5 as a white powder (35 mg, 61%). ¹H NMR (400 MHz, DMSO-d₆) δ 0.85 (t, J=6.6 Hz, 6H), 1.25 (br s, 50H), 1.55-1.65 (br s, 4H), 1.85-2.12 (br m, 10H), 2.88-3.05 (br m, 18H), 5.55 (br s, 1H), 7.85 (br s, 1H), 8.75-9.05 (br m, 4H); ¹³C NMR (100.6 MHz, DMSO-d₆) 13.1, 22.2, 22.42, 22.64, 28.6, 28.7, 29.0, 31.27, 36.18, 43.5, 43.8, 44.1, 54.4, 54.8, 130.7, 133.1; MS (FAB) calcd for C₄₅H₉₅N₅ (free amine) m/z 706.2 found 707.4 (MH)⁺.

The following references to the extent that they provide exemplary procedural or other details supplementary to those set forth herein, are specifically incorporated herein by reference.
Blackwell Publ., Determination of minimum inhibitory concentrations (MICs) of antibacterial agents by broth dilution, Clin. Microbiol. Infect. 9:1-7 (2003).
Anhalt, et al., Failure of Padac test strips to detect staphylococcal b-lactamase, Antimicrob. Agents Chemother. 21:993-994 (1982).
Bernard, et al., Phase separations induced by melittin in negatively-charged phospholipid bilayers as detected by fluorescence polarization and differential scanning calorimetry, Biochimica et Biophysica Acta 688:152-162 (1982).
Bhattacharjya, et al., Polymyxin B nonapeptide. Conformations in water and in the lipopolysaccharide-bound state determined by two-dimensional NMR and molecular dynamics, Biopolymers 41:251-265 (1997).
Blagbrough, I. S., A. J. Geall, and S. A. David, 2000. Lipopolyamines incorporating the teraamine spermine bound to an alkyl chain, sequester bacterial lipopolysaccharide. Bioorg. Med. Chem. Lett. 10:1959-1962.
Bolivar, F., R. L. Rodriguez, P. J. Greene, M. C. Betlach, H. L. Heynacker, H. W.
Boyer, J. H. Crosa, and S. Falkow. 1977. Construction and characterization of new cloning vehicles. II. A multipurpose cloning system. Gene 2:95-101.
David, S. A. 2001. Towards a rational development of anti-endotoxin agents: novel approaches to sequestration of bacterial endotoxins with small molecules. J. Molec. Recognition 14:370-387.
David, S. A., K. A. Balasubramanian, V. I. Mathan, and P. Balaram. 1992. Analysis of the binding of polymyxin B to endotoxic lipid A and core glycolipid using a fluorescent displacement probe. Biochim. Biophys. Acta 1165:147-152.
David, S. A., V. I. Mathan, and P. Balaram. 1992. Interaction of melittin with endotoxic lipid A. Biochim. Biophys. Acta 1123:269-274.
David, S. A., R. Silverstein, C. R. Amura, T. Kielian, and D. C. Morrison. 1999. Lipopolyamines: novel antiendotoxin compounds that reduce mortality in experimental sepsis caused by Gram negative bacteria. Antimicrob. Agents Chemother. 43:912-919.
Devine, D. A. and R. E. Hancock. 2002. Cationic peptides: distribution and mechanisms of resistance. Curr. Pharm. Des. 8:703-714.
Ding, B., Q. Guan, J. P. Walsh, J. S. Boswell, T. W. Winter, E. S. Winter, S. S. Boyd, C. Li, and P. B. Savage. 2002. Correlation of the antibacterial activities of cationic peptide antibiotics and cationic steroid antibiotics. J. Med. Chem. 45:663-659.
Ding, B., N. Yin, J. Cardenas-Garcia, R. Evanson, T. Orsak, M. Fan, G. Turin, and P. B. Savage. 2004. Origins of cell selectivity of cationic steroid antibiotics. J. Am. Chem. Soc. 126:13642-13648.
Fainerman, V. B. and R. Miller. 2004. Maximum bubble pressure tensiometry—an analysis of experimental constraints. Adv. Colloids Interface Sci. 108-109:287-301.
Fidai, S., S. W. Farmer, and R. E. Hancock. 1997. Interaction of cationic peptides with bacterial membranes. Methods Mol. Biol. 78:187-204.
Friedrich, C., M. G. Scott, N. Karunaratne, H. Yan, and R. E. Hancock. 1999. Salt-resistant alpha-helical cationic antimicrobial peptides. Antimicrob. Agents Chemother. 43:1542-1548.
Funahara, Y., and N. Hiroshi. 1980. Asymmetric localization of lipopolysaccharides on the outer membrane of salmonella typhymurium. J. Bacteriol. 141/3:1463-1465.
Hancock, R. E. 1997. Peptide antibiotics. Lancet 349:418-422.
Hancock, R. E. and D. S. Chapple. 1999. Peptide antibiotics. Antimicrob. Agents Chemother. 43:1317-1323.
Hancock, R. E. W. and P. G. W. Wong. 1984. Compounds which increase the permeability of the Pseudomonas aeruginosa outer membrane. Antimicrob. Agents Chemother. 26:48-52.
Houston-ME, J., L. H. Kondejewski, D. N. Karunaratne, M. Gough, S. Fidai, R. S. Hodges, and R. E. Hancock. 1998. Influence of preformed alpha-helix and alpha-helix induction on the activity of cationic antimicrobial peptides. J. Pept. Res. 52:81-88.
Hurley, J. C. 1992. Antibiotic-induced release of endotoxin: A reappraisal. Clin. Infect. Dis. 15:840-854.
Hurley, J. C. 1995. Antibiotic-induced release of endotoxin. A therapeutic paradox. Drug Saf. 12:183-195.
Jackson, J. J., H. Kropp, and J. C. Hurley. 1994. Influence of antibiotic class and concentration on the percentage of release of lipopolysaccharide from Escherichia coli. J. Infect. Dis. 169:471-473.
Lehrer, R. I., A. Barton, K. A. Daher, S. S. Harwig, T. Ganz, and M. E. Selsted. 1989. Interaction of human defensins with Escherichia coli. Mechanism of bactericidal activity. J. Clin. Invest. 84:553-561.
Lehrer, R. I., A. Barton, and T. Ganz. 1988. Concurrent assessment of inner and outer membrane permeabilization and bacteriolysis in E. coli by multiple-wavelength spectrophotometry. J. Immunol. Methods 108:153-158.
Li, C., L. P. Budge, C. D. Driscoll, B. M. Willardson, G. W. Allman, and P. B. Savage. 1999. Incremental conversion of outer-membrane permeabilizers into potent antibiotics for Gram negative bacteria. J. Am. Chem. Soc. 121:931-940.
Mayo, K. H., J. Haseman, H. C. Young, and J. W. Mayo. 2000. Structure-function relationships in novel peptide dodecamers with broad-spectrum bactericidal and endotoxin-neutralizing activities. Biochem. J. 349:717-728.
Menger, F. M. and J. S. Keiper. 2000. Gemini surfactants. Angew. Chem. Int. Ed. Engl. 39:1906-1920.
Miller, K. A., E. V. K. Suresh Kumar, S. J. Wood, J. R. Cromer, A. Datta, and S. A. David. 2005. Lipopolysaccharide Sequestrants: Structural Correlates of Activity and Toxicity in Novel Acylhomospermines. J. Med. Chem. 48:2589-2599.
Morrison, D. C. and D. M. Jacobs. 1976. Binding of polymyxin B to the lipid A portion of bacterial lipopolysaccharides. Immunochemistry 13:813-818.
Nagini, S. and S. Selvam. 1997. Biochemical indicators of membrane damage in the plasma and erythrocytes of rats fed the peroxisome proliferator di(2-ethylhexyl)phthalate. Med. Sci. Res. 25:119-121.
Nikaido, H. 1988. Bacterial resistance to antibiotics as a function of outer membrane permeability. Journal of Antimicrobial Therapy 22:17-22.
Nikaido, H. and M. Vaara. 1985. Molecular basis of bacterial outer membrane permeability. Microbiol. Rev. 49:1-32.
Novo, D. J., N. G. Perlmutter, R. H. Hunt, and H. M. Shapiro. 2000. Multiparameter flow cytometric analysis of antibiotic effects on membrane potential, membrane permeability, and bacterial counts of Staphylococcus aureus and Microccocus luteus. Antimicrob. Agents Chemother. 44:827-834.
Osborn, M. J. 1979. Biosynthesis and assembly of the lipopolysaccharide of the outer membrane, p. 15-34. In: M. Inouye (ed.), Bacterial outer membranes. Biogenesis and functions. John Wiley & Sons, New York, Chichester, Brisbane, Toronto.
Piers, K. L., M. H. Brown, and R. E. Hancock. 1994. Improvement of outer membrane-permeabilizing and lipopolysaccharide-binding activities of an antimicrobial cationic peptide by C-terminal modification. Antimicrob. Agents Chemother. 38:2311-2316.
Prins, J. M., M. A. van Agtmael, E. J. Kuijper, S. J. van Deventer, and P. Speelman. 1995. Antibiotic-induced endotoxin release in patients with Gram negative urosepsis: a double-blind study comparing imipenem and ceftazidime. J. Infect. Dis. 172:886-891.
Prins, J. M., S. J. H. Van Deventer, E. J. Kuijper, and P. Speelman. 1994. Clinical relevance of antibiotic-induced endotoxin release. Antimicrob. Agents Chemother. 38:1211-1218.
Rietschel, E. T., T. Kirikae, F. U. Schade, U. Mamat, G. Schmidt, H. Loppnow, A. J. Ulmer, U. Zahringer, U. Seydel, F. Di Padova, and a. et. 1994. Bacterial endotoxin: molecular relationships of structure to activity and function. FASEB J. 8:217-225.
Rosenthal, K. S. and D. R. Storm. 1977. Disruption of the Escherichia coli outer membrane permeability barrier by immobilized polymyxin B. The Journal of Antibiotics 30:1087-1092.
Ross, B. P., A. C. Braddy, R. P. McGeary, J. T. Blanchfield, L. Prozkai, and I. Toth. 2004. Micellar aggregation and membrane partitioning of bile salts, fatty acids, sodium dodecyl sulfate, and sugar-conjugated fatty acids: correlation with hemolytic potency and implications for drug delivery. Mol. Pharmaceutics 1:233-245.
Schindler, P. R. and M. Teuber. 1975. Action of polymyxin B on bacterial membranes: Morphological changes in the cytoplasm and in the outer membrane of Salmonella typhimurium and Escherichia coli B. Antimicrobial Agents and Chemotherapy 8:95-104.
Schmidt, E. J., J. S. Boswell, A. Walsh, M. M. Schellenberg, T. W. Winter, C. Li, G. W. Allman, and P. B. Savage. 2001. Activities of cholic acid-derived antimicrobial agents against multidrug-resistant bacteria. J. Antimicrob. Chemother. 47:671-674.
Scott, M. G., M. R. Gold, and R. E. Hancock. 1999. Interaction of cationic peptides with lipoteichoic acid and Gram positive bacteria. Infect. Immun. 67:6445-6453.
Scott, M. G., A. C. Vreugdenhil, W. A. Buurman, R. E. Hancock, and M. R. Gold. 2000. Cationic antimicrobial peptides block the binding of lipopolysaccharide (LPS) to LPS binding protein. J. Immunol. 164:549-553.
Skeriavaj, B., D. Romeo, and R. Gennaro. 1990. Rapid membrane permeabilization and inhibition of vital functions of Gram negative bacteria by bactenecins. Infect. Immun. 58:3724-3730.
Snyder, D. S. and T. J. McIntosh. 2000. The lipopolysaccharide barrier: correlation of antibiotic susceptibility with antibiotic permeability and fluorescent probe binding kinetics. Biochemistry 39:11777-11787.
Storm, D. R. and K. Rosenthal. 1977. Polymyxin and related peptide antibiotics. Annual Reviews of Biochemistry 46:723-763.
Strom, M. B., B. E. Haug, M. L. Skar, W. Stensen, T. Stiberg, and J. S. Svendsen. 2003. The pharmacophore of short cationic antibacterial peptides. J. Med. Chem. 46:1567-1570.
Thomas, C. J., N. Surolia, and A. Surolia. 1999. Surface plasmon resonance studies resolve the enigmatic endotoxin neutralizing activity of polymyxin B. J. Biol. Chem. 274:29624-29627.
Tsubery, H., I. Ofek, S. Cohen, and M. Fridkin. 2000. Structure activity relationship study of polymyxin B nonapeptide. Adv. Exp. Med. Biol. 479:219-222.
Tsubery, H., I. Ofek, S. Cohen, and M. Fridkin. 2000. Structure-function studies of polymyxin B nonapeptide: implications to sensitization of Gram negative bacteria. J. Med. Chem. 43:3085-3092.
Vaara, M. 1992. Agents That Increase the Permeability of the Outer Membrane. Microbiological Reviews 56:395-411.
Vaara, M. 1993. Antibiotic-supersusceptible mutants of Escherichia coli and Salmonella typhimurium. Antimicrob. Agents Chemother. 37:2255-2260.
Vaara, M. and M. Porro. 1996. Group of peptides that act synergistically with hydrophobic antibiotics against Gram negative enteric bacteria [published erratum appears in Antimicrob Agents Chemother 1997 February; 41(2):496]. Antimicrob. Agents Chemother. 40:1801-1805.
Vaara, M. and T. Vaara. 1983. Polycations as outer membrane disorganizing agents. Antimicrobial Agents and Chemotherapy 24:114-122.
Vaara, M. and T. Vaara. 1983. Polycations sensitize enteric bacteria to antibiotics. Antimicrobial Agents and Chemotherapy 24:107-113.
Vaara, M. and T. Vaara. 1983. Sensitization of Gram negative bacteria to antibiotics and complement by a nontoxic oligopeptide. Nature 303:526-528.
van't Hof, W., E. C. I. Veerman, E. J. Helmerhorst, and A. V. N. Amerongen. 2001. Antimicrobial peptides: properties and applicability. Biol. Chem. 382:597-619.
Viljanen, P., H. Matsunaga, Y. Kimura, and M. Vaara. 1991. The outer membrane permeability-increasing action of deacylpolymyxins. J. Antibiot. Tokyo. 44:517-523.
Wiese, A., M. Munstermann, T. Gutsmann, B. Lindner, K. Kawahara, U. Zahringer, and U. Seydel. 1998. Molecular mechanisms of polymyxin B-membrane interactions: direct correlation between surface charge density and self-promoted transport. J. Membr. Biol. 162:127-138.
Yasuda, K., C. Ohmizo, and T. Katsu. 2004. Mode of action of novel polyamines increasing the permeability of bacterial outer membrane. Int. J. Antimicrob. Agents 24:67-71.
Zhang, L., P. Dhillon, H. Yan, S. Farmer, and R. E. Hancock. 2000. Interactions of bacterial cationic peptide antibiotics with outer and cytoplasmic membranes of Pseudomonas aeruginosa. Antimicrob. Agents Chemother. 44:3317-3321.
Zorko, M., A. Majerle, D. Sarlah, M. M. Keber, B. Mohar, and R. Jerala. 2005. Combination of Antimicrobial and Endotoxin-Neutralizing Activities of Novel Oleoylamines. Antimicrob. Agents Chemother. 49:2307-2313.

From the foregoing it will be seen that this invention is one well adapted to attain all ends and objectives herein-above set forth, together with the other advantages which are obvious and which are inherent to the invention. Since many possible embodiments may be made of the invention without departing from the scope thereof, it is to be understood that all matters herein set forth or shown in the accompanying drawings are to be interpreted as illustrative, and not in a limiting sense. While specific embodiments have been shown and discussed, various modifications may of course be made, and the invention is not limited to the specific forms or arrangement of parts and steps described herein, except insofar as such limitations are included in the following claims. Further, it will be understood that certain features and subcombinations are of utility and may be employed without reference to other features and subcombinations. This is contemplated by and is within the scope of the claims.

What is claimed and desired to be secured by Letters Patent is as follows:

1. A method for treating a bacterial infection in a subject, comprising co-administering to a subject suffering from said infection an antibacterial agent and a sensitizing compound, wherein said sensitizing compound increases the susceptibility of a bacterium to said antibacterial agent, and wherein said sensitizing compound has a structure according to: 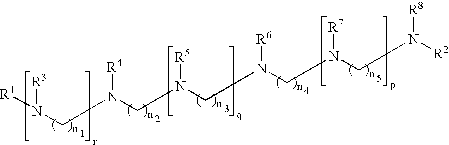
wherein R¹ and R² are independently hydrogen, C₇ to C₃₀ alkyl, C₇ to C₃₀ alkenyl, or C₇ to C₃₀ acyl; and wherein at least one of R¹ and R² is not hydrogen;
wherein R³, R⁴, R⁵, R⁶, R⁷, and R⁸ are independently hydrogen or lower alkyl;
wherein n₁, n₂, n₃, n₄, and n₅ are independently 1, 2, 3, 4, 5, 6, 7, 8, 9, or 10; and
wherein p, q, and r are independently 0, 1, 2, 3, 4, or 5;
and pharmaceutically acceptable salts thereof.

2. The method of claim 1 wherein said sensitizing compounds are polyamines characterized according to: 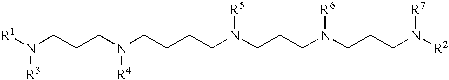
wherein R¹ and R² are independently hydrogen, C₇ to C₃₀ alkyl, C₇ to C₃₀ alkenyl, or C₇ to C₃₀ acyl; and wherein at least one of R¹ and R² is not hydrogen; and
wherein R³, R⁴, R⁵, R⁶, and R⁷ are independently hydrogen or lower alkyl;
and pharmaceutically acceptable salts thereof.

3. The method of claim 2 wherein R¹ and R² are independently acyl and selected from the group consisting of —COC₈H₁₇, —COC₉H₁₉, —COC₁₀H₂₁, —COC₁₁H₂₃, —COC₁₂H₂₅, —COC₁₃H₂₇, —COC₁₄H₂₉, —COC₁₅H₃₁, —COC₁₆H₃₃, —COC₁₇H₃₅, and —COC₁₈H₃₇.

4. The method of claim 2 wherein R¹ is hydrogen and wherein R² is an acyl selected from the group consisting of —COC₈H₁₇, —COC₉H₁₉, —COC₁₀H₂₁, —COC₁₁H₂₃, —COC₁₂H₂₅, —COC₁₃H₂₇, —COC₁₄H₂₉, —COC₁₅H₃₁, —COC₁₆H₃₃, —COC₁₇H₃₅, and —COC₁₈H₃₇.

5. The method of claim 2 wherein the sensitizing agent is defined according to:
NH₂C₃H₆NHC₄H₈NHC₃H₆NHC₃H₆NH—CO(CH₂)ₓCH₃
or
CH₃(CH₂)ₓCO—NHC₃H₆NHC₄H₈NHC₃H₆NHC₃H₆NH—CO(CH₂)ₓCH₃
wherein x is an integer between 7 and 25;
and pharmaceutically acceptable salts thereof.

6. The method of claim 5 wherein x is between 10 and 18.

7. The method of claim 2 wherein R¹ and R² are independently a C₇ to C₃₀ alkyl.

8. The method of claim 2 wherein R¹ is hydrogen and wherein R² is a C₇ to C₃₀ alkyl.

9. The method of claim 2 where the sensitizing agent is defined according to:
CH₃(CH₂)ₓ—NHC₃H₆NHC₄H₈NHC₃H₆NHC₃H₆NH—(CH₂)ₓCH₃
or
NH₂C₃H₆NHC₄H₈NHC₃H₆NHC₃H₆NH—(CH₂)ₓCH₃
wherein x is an integer between 7 and 29;
and pharmaceutically acceptable salts thereof.

10. The method of claim 2 wherein R³, R⁴, R⁵, R⁶ and R⁷ are all hydrogen according to:
NH₂C₃H₆NHC₄H₈NHC₃H₆NHC₃H₆NH—R²
and wherein R² is C₇ to C₃₀ alky;
and pharmaceutically acceptable salts thereof

11. The method of claim 2 wherein R¹ and R² are independently C₇ to C₃₀ alkenyl.

12. The method of claim 2 wherein R¹ is hydrogen and wherein R² is a C₇ to C₃₀ alkenyl.

13. The method of claim 2 where the sensitizing agent is defined according to:
NH₂C₃H₆NHC₄H₈NHC₃H₆NHC₃H₆NH—(CH₂)C(R¹⁰)═CHR¹¹
or
R¹¹═CHC(R¹⁰)CH₂—NH₂C₃H₆NHC₄H₈NHC₃H₆NHC₃H₆NH—CH₂)C(R¹⁰)═CHR¹¹
wherein R¹⁰ and R¹¹ are independently C₇ to C₂₉ alkyl;
and pharmaceutically acceptable salts thereof.

14. The method of claim 2 wherein R³, R⁴, R⁵, R⁶, and R⁷ are all hydrogen according to:
NH₂C₃H₆NHC₄H₈NHC₃H₆NHC₃H₆NH—R²
wherein R² is a C₇ to C₃₀ alkenyl;
and pharmaceutically acceptable salts thereof.

15. The method of claim 1 wherein said sensitizing compounds are polyamines characterized according to: 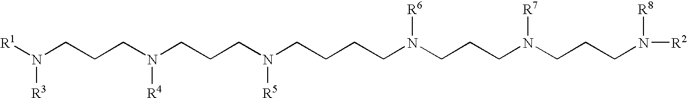
wherein R¹ and R² are independently hydrogen, C₇ to C₃₀ alkyl, C₇ to C₃₀ alkenyl, or C₇ to C₃₀ acyl; and wherein at least one of R¹ and R² is not hydrogen; and
wherein R³, R⁴, R⁵, R⁶, R⁷, and R⁸ are independently hydrogen or lower alkyl;
and pharmaceutically acceptable salts thereof.

16. The method of claim 15 wherein R¹ and R² are independently acyl and selected from the group consisting of —COC₈H₁₇, —COC₉H₁₉, —COC₁₀H₂₁, —COC₁₁H₂₃, —COC₁₂H₂₅, —COC₁₃H₂₇, —COC₁₄H₂₉, —COC₁₅H₃₁, —COC₁₆H₃₃, —COC₁₇H₃₅, and —COC₁₈H₃₇.

17. The method of claim 15 wherein R¹ is hydrogen and wherein R² is an acyl selected from the group consisting of —COC₈H₁₇, —COC₉H₁₉, —COC₁₀H₂₁, —COC₁₁H₂₃, —COC₁₂H₂₁, —COC₁₃H₂₇, —COC₁₄H₂₉, —COC₁₅H₃₁, —COC₁₆H₃₃, —COC₁₇H₃₅, and —COC₁₈H₃₇.

18. The method of claim 15 wherein the sensitizing agent is defined according to:
CH₃(CH₂)ₓCO—NHC₃H₆NHC₃H₆NHC₄H₈NHC₃H₆NHC₃H₆NH—CO(CH₂)ₓCH₃
or
NH₂C₃H₆NHC₃H₆NHC₄H₈NHC₃H₆NHC₃H₆NH—CO(CH₂)ₓCH₃
wherein x is an integer between 7 and 25;
and pharmaceutically acceptable salts thereof.

19. The method of claim 18 wherein x is between 8 and 13.

20. The method of claim 15 wherein R¹ and R² are independently C₇ to C₃₀ alkyl.

21. The method of claim 15 wherein R¹ is hydrogen and wherein R² is a C₇ to C₃₀ alkyl.

22. The method of claim 15 wherein the sensitizing agent is defined according to:
NH₂C₃H₆NHC₃H₆NHC₄H₈NHC₃H₆NHC₃H₆NH—(CH₂)ₓCH₃
or
CH₃(CH₂)ₓ—NHC₃H₆NHC₃H₆NHC₄H₈NHC₃H₆NHC₃H₆NH—(CH₂)ₓCH₃
wherein x is an integer between 7 and 29;
and pharmaceutically acceptable salts thereof.

23. The method of claim 15 wherein R³, R⁴, R⁵, R⁶, R⁷, and R⁸ are all hydrogen according to:
NH₂C₃H₆NHC₃H₆NHC₄H₈NHC₃H₆NHC₃H₆NH—R²
and wherein R² is C₇ to C₃₀ alkyl;
and pharmaceutically acceptable salts thereof.

24. The method of claim 15 wherein R¹ and R² are independently C₇ to C₃₀ alkenyl.

25. The method of claim 15 wherein R¹ is hydrogen and wherein R² is a C₇ to C₃₀ alkenyl.

26. The method of claim 15 wherein the sensitizing agent is defined according to:
NH₂C₃H₆NHC₃H₆NHC₄H₈NHC₃H₆NHC₃H₆NH—(CH₂)C(R¹⁰)═CHR¹¹
or
R¹¹═CHC(R¹⁰)CH₂—NHC₃H₆NHC₃H₆NHC₄H₈NHC₃H₆NHC₃H₆NH—(CH₂)C(R¹⁰)═CHR¹¹
wherein R¹⁰ and R¹¹ are independently C₇ to C₂₀ alkyl;
and pharmaceutically acceptable salts thereof.

27. The method of claim 15 wherein R¹, R³, R⁴, R⁵, R⁶, R⁷, and R⁸ are all hydrogen according to:
NH₂C₃H₆NHC₃H₆NHC₄H₈NHC₃H₆NHC₃H₆NH—R²
and wherein R² is a C₇ to C₃₀ alkenyl;
and pharmaceutically acceptable salts thereof.

28. The method of claim 1 wherein said sensitizing agent is selected from the group consisting of NH₂C₃H₆NHC₄H₈NHC₃H₆NHC₃H₆NHC₁₆H₃₃ and (DS-96); NH₂C₃H₆NHC₄H₈NHC₃H₆NHC₃H₆NHCHC(C₁₄H₂₉)═C₁₆H₃₂ (EVK-203) and pharmaceutically acceptable salts thereof.

29. The method of claim 1, wherein said antibacterial agent is selected from the group consisting of glycopeptides, macrolides, quinolones, tetracyclines, and aminoglycosides.

30. The method of claim 1, wherein said antibacterial agent is a beta-lactam.

31. The method of claim 30, wherein said beta-lactam is selected from the group consisting of ampicillin, amoxicillin, cloxacillin, flucloxacillin, methicillin, oxacillin, piperacillin, azlocillin, mezlocillin, cefaclor, cefalexin, cefamandole, cefazolin, cefonicid, cefoperazone, cefotaxime, cefoxitin, ceftazidime, cefpirome, ceftriaxone, cephalothin, ceftibuten, cefixime, cefpodoxime, loracarbef, imipenem and meropenem.

32. The method of claim 1, wherein said sensitizing agent also has intrinsic antibacterial activity.

33. The method of claim 1 wherein said bacteria is a Gram negative bacteria.

34. The method of claim 1 wherein said sensitizing compound is administered intravenously.

35. The method of claim 34 wherein said sensitizing compound is complexed with albumin.

36. A pharmaceutical composition effective for treatment of an infection of a subject by bacteria, comprising a sensitizing compound and an antibacterial agent, wherein said sensitizing compound has a structure of: 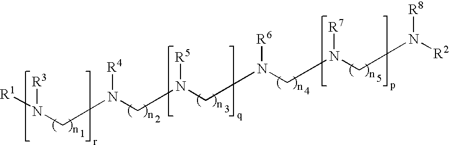
wherein R¹ and R² are independently hydrogen, C₇ to C₃₀ alkyl, C₇ to C₃₀ alkenyl, or C₇ to C₃₀ acyl; and wherein at least one of R¹ and R² is not hydrogen;
wherein R³, R⁴, R⁵, R⁶, R⁷, and R⁸ are independently hydrogen or lower alkyl;
wherein n₁, n₂, n₃, n₄, and n₅ are independently 1, 2, 3, 4, 5, 6, 7, 8, 9, or 10; and
and wherein p, q, and r are independently 0, 1, 2, 3, 4, or 5;
and pharmaceutically acceptable salts thereof

37. The composition of claim 36, wherein said antibacterial agent is selected from the group consisting of glycopeptides, macrolides, quinolones, tetracyclines, and aminoglycosides.

38. The composition of claim 36, wherein said antibacterial agent is a beta-lactam.

39. The composition of claim 38, wherein said beta-lactam is selected from the group consisting of ampicillin, amoxicillin, cloxacillin, flucloxacillin, methicillin, oxacillin, piperacillin, azlocillin, mezlocillin, cefaclor, cefalexin, cefamandole, cefazolin, cefonicid, cefoperazone, cefotaxime, cefoxitin, ceftazidime, cefpirome, ceftriaxone, cephalothin, ceftibuten, cefixime, cefpodoxime, loracarbef, imipenem and meropenem.

40. The composition of claim 36 further comprising a carrier.

41. Compounds according to: 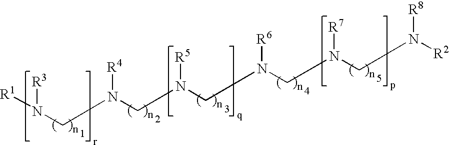
wherein R¹ and R² are independently hydrogen, C₇ to C₃₀ alkyl or C₇ to C₃₀ alkenyl; and
wherein at least one of R¹ and R² is not hydrogen;
wherein R³, R⁴, R⁵, R⁶, R⁷, and R⁸ are independently hydrogen or lower alkyl;
wherein n₁, n₂, n₃, n₄, and n₅ are independently 1, 2, 3, 4, 5, 6, 7, 8, 9, or 10; and
and wherein p, q, and r are independently 0, 1, 2, 3, 4, or 5;
and pharmaceutically acceptable salts thereof.

42. The compounds according to claim 41 according to 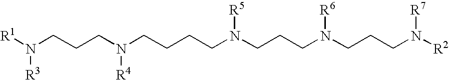
wherein R¹ and R² are independently hydrogen, C₇ to C₃₀ alkyl or C₇ to C₃₀ alkenyl, and
wherein at least one of R¹ and R² is not hydrogen; and
wherein R³, R⁴, R⁵, R⁶, and R⁷ are independently hydrogen or lower alkyl;
and pharmaceutically acceptable salts thereof.

43. The compounds according to claim 42 wherein R¹ and R² are independently a C₇ to C₃₀ alkyl.

44. The compounds according to claim 42 wherein R¹ is hydrogen and wherein R² is a C₇ to C₃₀ alkyl.

45. The compounds according to claim 42 according to:
CH₃(CH₂)ₓ—NHC₃H₆NHC₄H₈NHC₃H₆NHC₃H₆NH—(CH₂)ₓCH₃
or
NH₂C₃H₆NHC₄H₈NHC₃H₆NHC₃H₆NH—(CH₂)ₓCH₃
wherein x is an integer between 7 and 29;
and pharmaceutically acceptable salts thereof.

46. The compounds according to claim 42 wherein R³, R⁴, R⁵, R⁶, and R⁷ are all hydrogen according to:
NH₂C₃H₆NHC₄H₈NHC₃H₆NHC₃H₆NH—R²
and wherein R² is C₇ to C₃₀ alkyl;
and pharmaceutically acceptable salts thereof.

47. The compounds according to claim 42 wherein R¹ and R² are independently C₇ to C₃₀ alkenyl.

48. The compounds according to claim 42 wherein R¹ is hydrogen and wherein R² is a C₇ to C₃₀ alkenyl.

49. The compounds according to claim 42 according to:
NH₂C₃H₆NHC₄H₈NHC₃H₆NHC₃H₆NH—(CH₂)C(R¹⁰)═CHR¹¹
or
R¹¹═CHC(R¹⁰)CH₂—NH₂C₃H₆NHC₄H₈NHC₃H₆NHC₃H₆NH—(CH₂)C(R¹⁰)═CHR¹¹
wherein R¹⁰ and R¹¹ are independently C₇ to C₂₀ alkyl;
and pharmaceutically acceptable salts thereof.

50. The compounds according to claim 42 wherein R³, R⁴, R⁵, R⁶, and R⁷ are all hydrogen according to:
NH₂C₃H₆NHC₄H₈NHC₃H₆NHC₃H₆NH—R²
wherein R² is a C₇ to C₃₀ alkenyl;
and pharmaceutically acceptable salts thereof.

51. The compounds according to claim 41 according to: 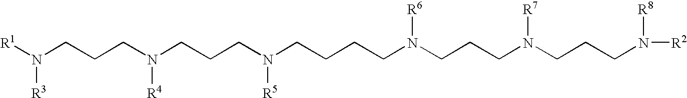
wherein R¹ and R² are independently hydrogen, C₇ to C₃₀ alkyl or C₇ to C₃₀ alkenyl, and
wherein at least one of R¹ and R² is not hydrogen; and
wherein R³, R⁴, R⁵, R⁶, R⁷, and R⁸ are independently hydrogen or lower alkyl;
and pharmaceutically acceptable salts thereof.

52. The compounds according to claim 51 wherein R¹ and R² are independently C₇ to C₃₀ alkyl.

53. The compounds according to claim 51 wherein R¹ is hydrogen and wherein R² is a C₇ to C₃₀ alkyl.

54. The compounds according to claim 51 according to:
NH₂C₃H₆NHC₃H₆NHC₄H₈NHC₃H₆NHC₃H₆NH—(CH₂)ₓCH₃
or
CH₃(CH₂)ₓ—NHC₃H₆NHC₃H₆NHC₄H₈NHC₃H₆NH₃H₆NH—(CH₂)ₓCH₃
wherein x is an integer between 7 and 29;
and pharmaceutically acceptable salts thereof.

55. The compounds according to claim 51 wherein R³, R⁴, R⁵, R⁶, R⁷, and R⁸ are all hydrogen according to:
NH₂C₃H₆NHC₃H₆NHC₄H₈NHC₃H₆NHC₃H₆NH—R²
and wherein R² is C₇ to C₃₀ alkyl;
and pharmaceutically acceptable salts thereof.

56. The compounds according to claim 51 wherein R¹ and R² are independently C₇ to C₃₀ alkenyl.

57. The compounds according to claim 51 wherein R¹ is hydrogen and wherein R² is a C₇ to C₃₀ alkenyl.

58. The compounds according to claim 51 according to:
NH₂C₃H₆NHC₃H₆NHC₄H₈NHC₃H₆NHC₃H₆NH—(CH₂)C(R¹⁰)═CHR¹¹
or
R¹¹═CHC(R¹⁰)CH₂—NHC₃H₆NHC₃H₆NHC₄H₈NHC₃H₆NHC₃H₆NH—(CH₂)C(R¹⁰)═CHR¹¹
wherein R¹⁰ and R¹¹ are independently C₇ to C₂₀ alkyl;
and pharmaceutically acceptable salts thereof.

59. The method of claim 51 wherein R¹, R³, R⁴, R⁵, R⁶, R⁷, and R⁸ are all hydrogen according to:
NH₂C₃H₆NHC₃H₆NHC₄H₈NHC₃H₆NHC₃H₆NH—R²
and wherein R² is a C₇ to C₃₀ alkenyl;
and pharmaceutically acceptable salts thereof.

60. The compounds according to claim 41 selected from the group consisting of NH₂C₃H₆NHC₄H₈NHC₃H₆NHC₃H₆NHC₁₆H₃₃ and (DS-96);
NH₂C₃H₆NHC₄H₈NHC₃H₆NHC₃H₆NHCHC(C₁₄H₂₉)═C₁₆H₃₂ (EVK-203) and pharmaceutically acceptable salts thereof.

61. A pharmaceutical composition comprising a therapeutically effective amount of a compound according to claim 41 and a pharmaceutically acceptable carrier.

62. The pharmaceutical composition of claim 61 wherein said carrier is albumin.
